SEQUENCE LISTING (22 sequences)

SEQ ID NO: 1 - DNA, 10 bp, Artificial Sequence
  Primer for random amplified polymorphic DNA
ggtgcgggaa

SEQ ID NO: 2 - DNA, 10 bp, Artificial Sequence
  Primer for random amplified polymorphic DNA
gtttcgctcc

SEQ ID NO: 3 - DNA, 10 bp, Artificial Sequence
  Primer for random amplified polymorphic DNA
gtagacccgt

SEQ ID NO: 4 - DNA, 10 bp, Artificial Sequence
  Primer for random amplified polymorphic DNA
aagagcccgt

SEQ ID NO: 5 - DNA, 10 bp, Artificial Sequence
  Primer for random amplified polymorphic DNA
aacgcgcaac

SEQ ID NO: 6 - DNA, 10 bp, Artificial Sequence
  Primer for random amplified polymorphic DNA
cccgtcagca

SEQ ID NO: 7 - DNA, 20 bp, Artificial Sequence
  Primer for PCR amplification of 16S ribosomal RNA gene
agagtttgatymtggctcag

SEQ ID NO: 8 - DNA, 20 bp, Artificial Sequence
  Primer for PCR amplification of 16S ribosomal RNA gene
actaccrgggtatctaatcc

SEQ ID NO: 9 - DNA, 20 bp, Artificial Sequence
  Primer set for cell culture screening
gccacctcagacaaaatgct

SEQ ID NO: 10 - DNA, 20 bp, Artificial Sequence
  Primer for cell culture screening
tctgccagtacctccttgct

SEQ ID NO: 11 - DNA, 21 bp, Artificial Sequence
  Primer for cell culture screening
acttccaaactggctgttgcc

SEQ ID NO: 12 - DNA, 21 bp, Artificial Sequence
  Primer for cell culture screening
ctgctgttgttgttgcttctc

SEQ ID NO: 13 - DNA, 20 bp, Artificial Sequence
  Primer for cell culture screening
cccaaggactcagatcatcg

SEQ ID NO: 14 - DNA, 21 bp, Artificial Sequence
  Primer for cell culture screening
ataccccactctgccattgga

SEQ ID NO: 15 - DNA, 20 bp, Artificial Sequence
  Primer for cell culture screening
ggacctgaccgactacctca

SEQ ID NO: 16 - DNA, 20 bp, Artificial Sequence
  Primer for cell culture screening
gcgacgtagcagagcttctc

SEQ ID NO: 17 - DNA, 22 bp, Artificial Sequence
  Primer for microbial screening
ggcagccttgtcccttgaagag

SEQ ID NO: 18 - DNA, 22 bp, Artificial Sequence
  Primer for microbial screening
gtagcccacgtcgtagcaaacc

SEQ ID NO: 19 - DNA, 20 bp, Artificial Sequence
  Primer for microbial screening
gcaaggctaactgacctgga

SEQ ID NO: 20 - DNA, 20 bp, Artificial Sequence
  Primer for microbial screening
ctttgattctgcccgttgag

SEQ ID NO: 21 - DNA, 20 bp, Artificial Sequence
  Primer for microbial screening
tgacgaggcccagagcaaga

SEQ ID NO: 22 - DNA, 20 bp, Artificial Sequence
  Primer for microbial screening
atgggcacagtgtgggtgac

(19) United States
(12) Patent Application Publication
(10) Pub. No.: US 2013/0045185 A1
(43) Pub. Date: Feb. 21, 2013

(54) STRAINS AND METHODS USEFUL FOR MYCOTOXINS

(71) Applicants: Mari Ellen Davis, Waukesha, WI (US); Joshua Rehberger, Milwaukee, WI (US); Kimberly Novak, New Berlin, WI (US); Thomas G. Rehberger, Wauwatosa, WI (US)

(72) Inventors: Mari Ellen Davis, Waukesha, WI (US); Joshua Rehberger, Milwaukee, WI (US); Kimberly Novak, New Berlin, WI (US); Thomas G. Rehberger, Wauwatosa, WI (US)

(73) Assignee: DUPONT NUTRITION BIOSCIENCES APS, Copenhagen (DK)

(21) Appl. No.: 13/588,794

(22) Filed: Aug. 17, 2012

Related U.S. Application Data
(60) Provisional application No. 61/525,033, filed on Aug. 18, 2011.

(51) Int. Cl. C12N 1/20; A61P 37/02; A61K 35/74; A61P 1/00

(52) U.S. Cl. 424/93.3; 435/252.5; 435/252.9; 435/252.1; 435/252.4; 424/115

(57) ABSTRACT

The disclosure relates to strains, compositions and methods for detoxifying a mycotoxin. In another embodiment, the disclosure relates to strains, compositions and methods for alleviating the gastrointestinal inflammatory response resulting from ingestion of mycotoxins. In one embodiment, the strains are Bacillus strains. In another embodiment, the strains are lactic acid bacteria.

### CROSS-REFERENCE TO RELATED APPLICATIONS

This application claims priority under 35 U.S.C. §119(e) to U.S. Provisional Patent Application No. 61/525,033 filed Aug. 18, 2011, the entirety of which is incorporated by reference herein.

### BIBLIOGRAPHY

Complete bibliographic citations of the references referred to herein by the first author's last name in parentheses can be found in the Bibliography section, immediately preceding the claims.

### FIELD

The invention relates to strains and methods for detoxifying mycotoxins. The invention also is related to strains and methods for alleviating the gastrointestinal inflammatory response resulting from ingestion of mycotoxins. In addition, the invention relates to methods for identifying strains that detoxify mycotoxins and/or reduce inflammatory effects of mycotoxins. The invention also relates to methods for reducing mold growth.

### BACKGROUND

Mycotoxin contamination of grain sources in livestock diets results in significant economic loss due to decreases in animal production performance. An annual report by Biomin on a survey of the prevalence of mycotoxins in cereal grains over the course of the 2010 year indicated that grain contamination by mycotoxins was ubiquitous all over the world and that the most prevalent contaminates specifically were the fumonisin and deoxynivalenol (DON) mycotoxins. DON mycotoxins are also known as food refusal factor, emetic factor, and vomitoxin. High concentrations of DON are of great concern to swine producers, as pigs seem to be very sensitive to this mycotoxin, exhibiting signs of vomiting, diarrhea, and decreased feed intake or feed refusal. Mycotoxin contamination of corn fed to swine will likely be a continued problem as more dried distiller's grains are fed to swine.

The majority of mycotoxins come from the fungi Aspergillus, Penicillium, and Fusarium (Bouhet and Oswald, 2005). These three fungi produce mycotoxins that negatively impact animal productivity. Farm management practices as well as the environment play a role in mycotoxin production and their exposure to livestock. Higher concentrations of mycotoxins are found in grain that is damaged, stored in a warm environment with high moisture, or stored in an environment where the temperature fluctuates between warm and cool, such as during the change of seasons (Osweiler, 2006).

Mycotoxins that tend to be problematic in swine production include aflatoxins, ochratoxin, citrinin, zearalenone, fumonisins, and trichothecenes (Bennett and Klich, 2003; Bouhet and Oswald, 2005; Osweiler, 2006). In pigs, trichothecenes cause feed refusal, gastroenteritis, diarrhea, skin irritation and necrosis, lymphatic tissue depletion, suppression of immune function, shock, cardiovascular failure and death.

Deoxynivalenol also triggers the transcription factors, nuclear factor-KB (NF-κB) and activator protein-1 (AP-1), involved in cell signaling pathways controlling the production of inflammatory cytokines (Pestka, 2003). These inflammatory cytokines modulate metabolic activity and are potent inhibitors of feed intake and accretion of protein tissue (Spurlock, 1997), and serve as an additional mechanism of action by which DON negatively impacts pig performance. Whereas specific inflammatory immune responses are enhanced by DON exposure, apoptotic effects of the trichothecenes result in immunosuppression and subsequent susceptibility to pathogen challenges (Bondy and Pestka, 2000).

Therefore, what is needed are strains and methods for detoxifying mycotoxins, strains and methods for alleviating the gastrointestinal inflammatory response resulting from ingestion of mycotoxins, methods for identifying strains that detoxify mycotoxins and/or reduce inflammatory effects of mycotoxins, and methods for reducing or inhibiting the amount of mold growth.

### SUMMARY

The disclosure relates to strains that are useful for detoxifying one or more mycotoxins. In one embodiment, the detoxifying strains include Bacillus strains, including, but not limited to, B. subtilis, B. licheniformis, B. pumilus, B. coagulans, B. amyloliquefaciens, B. stearothermophilus, B. brevis, B. alkalophilus, B. clausii, B. halodurans, B. megaterium, B. circulans, B. lautus, B. thuringiensis and B. lentus strains. In at least some embodiments, the B. subtilis strain(s) is (are) B. subtilis 4-7d and B. subtilis 3-5h. In at least some embodiments, the B. licheniformis strain(s) is (are) B. licheniformis 4-2a and B. licheniformis 3-12a.

In another embodiment, the disclosure relates to bacterial microorganisms that alleviate the gastrointestinal inflammatory response resulting from ingestion of one or more mycotoxin(s). In one embodiment, the bacterial microorganisms include but are not limited to lactic acid bacteria (LAB) and Bacillus strains. In another embodiment, the strains are L. johnsonii PLC B6 and E. faecium 2-1d.

In another embodiment, the disclosure relates to methods of culturing a microorganism including but not limited to lactic acid bacteria (LAB) and Bacillus strains.

In still another embodiment, the disclosure relates to a composition including one or more strain(s) described herein. The composition can be fed to an animal as a direct-fed microbial (DFM).

In yet another embodiment, the disclosure relate to an animal feed. In another embodiment, the animal feed is supplemented with a bacterial strain capable of detoxifying one or more mycotoxin(s). In another embodiment, the bacterial strain is lactic acid bacteria (LAB) and Bacillus strains.

In another embodiment, the disclosure relates to an animal feed supplemented with a bacterial strain capable of alleviating a gastrointestinal inflammatory response. In one embodiment, the gastrointestinal inflammatory response is induced by a mycotoxin. In another embodiment, the bacterial strain is lactic acid bacteria (LAB) and Bacillus strains.

In another embodiment, the disclosure relates to methods comprising administering to an animal an effective amount of a strain capable of detoxifying one or more mycotoxin(s) and/or alleviating a gastrointestinal inflammatory response. In one embodiment, the strain includes but is not limited to a lactic acid bacteria (LAB) and Bacillus strains. In one embodiment, administering a lactic acid bacteria (LAB) and/or a Bacillus strain to an animal improves performance of the animal. Any performance indicator may be improved by the strains, compositions, and methods herein including but not limited to average daily feed intake (ADFI), average daily gain (ADG), or feed efficiency (gain:feed; G:F).

In one embodiment, the disclosure provides methods for detoxifying one or more mycotoxins. In one embodiment, the method for detoxifying a mycotoxin comprises administering to an animal an effective amount of a detoxifying strain or an anti-inflammatory strain or combinations thereof.

In yet another embodiment, the disclosure relates to methods for reducing mold growth. In one embodiment, the method comprises using lactic acid bacteria (LAB) and Bacillus strains. In another embodiment, the method for reducing mold growth comprises supplementing an animal feed with strain, a composition, supernatant or a combination of the aforementioned, wherein the strain is a lactic acid bacteria strain or a Bacillus strain. In one embodiment, the animal feed is a cereal grain.

In another embodiment, the disclosure relates to methods for identifying strains with desired characteristics.

### BRIEF DESCRIPTION OF THE DRAWINGS

Exemplary embodiments of the invention are illustrated in the accompanying drawings, in which like reference numerals represent like parts throughout and in which:

FIG. 1 is a diagram showing an embodiment of an in vitro screening procedure for identifying deoxynivalenol (DON) detoxifying microorganisms.

FIG. 2 is a diagram showing an embodiment of a procedure for selecting candidate microorganisms for anti-inflammatory effects in vitro.

FIG. 3 is diagram showing an example of a plate set up for candidate direct-fed microbial cell culture screening with deoxynivalenol.

FIG. 4 is a photograph showing Primer 2 and 3 RAPD PCR fingerprint images of Bacillus subtilis strain 4-7d isolates.

FIG. 5 shows partial 16S rDNA sequence of B. subtilis strain 4-7d.

FIG. 6 is a photograph showing Primer 2 and 3 RAPD PCR fingerprint images of B. subtilis strain 3-5h isolates.

FIG. 7 shows partial 16S rDNA sequence of B. subtilis strain 3-5h.

FIG. 8 is a photograph showing Primer 2 and 3 RAPD PCR fingerprint images of B. licheniformis strain 4-2a isolates.

FIG. 9 shows partial 16S rDNA sequence of B. licheniformis 4-2a.

FIG. 10 is a photograph showing Primer 2 and 3 RAPD PCR fingerprint images of B. licheniformis strain 3-12a isolates.

FIG. 11 shows partial 16S rDNA sequence of B. licheniformis 3-12a.

Before explaining embodiments of the invention in detail, it is to be understood that the invention is not limited in its application to the details of construction and the arrangement of the components set forth in the following description or illustrated in the drawings. The invention is capable of other embodiments or being practiced or carried out in various ways. Also, it is to be understood that the phraseology and terminology employed herein is for the purpose of description and should not be regarded as limiting.

### DETAILED DESCRIPTION

### Definitions

The numerical ranges in this disclosure are approximate, and thus may include values outside of the range unless otherwise indicated. Numerical ranges include all values from and including the lower and the upper values, in increments of one unit, provided that there is a separation of at least two units between any lower value and any higher value. As an example, if a compositional, physical or other property, such as, for example, molecular weight, viscosity, etc., is from 100 to 1,000, it is intended that all individual values, such as 100, 101, 102, etc., and sub ranges, such as 100 to 144, 155 to 170, 197 to 200, etc., are expressly enumerated. For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure. Numerical ranges are provided within this disclosure for, among other things, relative amounts of components in a mixture, and various temperature and other parameter ranges recited in the methods.

By “administer,” is meant the action of introducing at least one strain and/or supernatant from a culture of at least one strain described herein into the animal's gastrointestinal tract. More particularly, this administration is an administration by oral route. This administration can in particular be carried out by supplementing the feed intended for the animal with the at least one strain, the thus supplemented feed then being ingested by the animal. The administration can also be carried out using a stomach tube or any other way to make it possible to directly introduce the at least one strain into the animal's gastrointestinal tract.

By “at least one strain,” is meant a single strain but also mixtures of strains comprising at least two strains of bacteria. By “a mixture of at least two strains,” is meant a mixture of two, three, four, five, six or even more strains. In some embodiments of a mixture of strains, the proportions can vary from 1% to 99%. In certain embodiments, the proportion of a strain used in the mixture is at least 5%, 10%, 15%, 20%, 25%, 30%, 35%, 40%, 45%, 50%, 55%, 60%, 65%, 70%, 75%, 80%, 85%, 90%, or 95%. Other embodiments of a mixture of strains are from 25% to 75%. Additional embodiments of a mixture of strains are approximately 50% for each strain. When a mixture comprises more than two strains, the strains can be present in substantially equal proportions in the mixture or in different proportions.

By “effective amount,” is meant a quantity of DFM and/or supernatant sufficient to allow improvement, i.e., for the detoxifying strains, reduction in the amount of toxicity of a mycotoxin in comparison with a reference; for the anti-inflammatory strains, reduction in the amount of inflammation in the gastrointestinal tract of an animal to which the strain is administered; and for strains that reduce or inhibit mold growth, reduction or inhibition in the amount of mold growth as compared to a reference control. The mycotoxin reductive effect, the inflammation reductive amount, and the mold growth reductive effect can be measured as described herein or by other methods known in the art. These effective amounts can be administered to the animal by providing ad libitum access to feed containing the DFM. The DFM can also be administered in one or more doses.

As used herein, a “variant” has at least 80% identity of genetic sequences with the disclosed strains using random amplified polymorphic DNA polymerase chain reaction (RAPD-PCR) analysis. The degree of identity of genetic sequences can vary. In some embodiments, the variant has at least 85%, 90%, 95%, 96%, 97%, 98%, or 99% identity of genetic sequences with the disclosed strains using RAPD-PCR analysis. Six primers that can be used for RAPD-PCR analysis include the following: Primer 1 (5′-GGTGCGGGAA-3′) (SEQ ID NO. 1), PRIMER 2 (5′-GTTTCGCTCC-3′) (SEQ ID NO. 2), PRIMER 3 (5′-GTAGACCCGT-3′) (SEQ ID NO. 3), PRIMER 4 (5′-AAGAGCCCGT-3′) (SEQ ID NO. 4), PRIMER 5 (5′-AACGCGCAAC-3′) (SEQ ID NO. 5), PRIMER 6 (5′-CCCGTCAGCA-3′) (SEQ ID NO. 6). RAPD analysis can be performed using Ready-to-Go™ RAPD Analysis Beads (Amersham Biosciences, Sweden), which are designed as pre-mixed, pre-dispensed reactions for performing RAPD analysis.

Mycotoxin contamination of grain sources in livestock diets results in significant economic loss due to decreases in animal production performance. Mycotoxins include aflatoxins, ochratoxin, citrinin, zearalenone, fumonisins, and trichothecenes. T-2 toxin, diacetoxyscirpenol (DAS), and deoxynivalenol (DON) are trichothecenes. The mycotoxin DON is of great concern to swine producers, as pigs seem to be very sensitive to this mycotoxin. However, the strains and methods described and claimed herein are useful for other animals, including, but not limited to, those listed below. It has been found that certain live, spore-forming bacterial microorganisms reduce the toxicity of mycotoxins, including but not limited to, DON. In at least some embodiments, the detoxification is through transformation of the toxic compound to one substantially without toxic characteristics. These microorganisms are hereinafter referred to as “detoxifying strain(s).” These strains are useful for detoxifying DON and other mycotoxins. The detoxifying strain(s) can be fed to animals to detoxify mycotoxins present in animal feed. In some embodiments, the detoxifying strain(s) can be fed to animals to detoxify DON present in animal feed.

### Detoxifying Strains

The detoxifying strains include Bacillus strains, including, but not limited to, B. subtilis, B. licheniformis, B. pumilus, B. coagulans, B. amyloliquefaciens, B. stearothermophilus, B. brevis, B. alkalophilus, B. clausii, B. halodurans, B. megaterium, B. circulans, B. lautus, B. thuringiensis and B. lentus strains. In at least some embodiments, the B. subtilis strain(s) is (are) B. subtilis 4-7d and B. subtilis 3-5h. In at least some embodiments, the B. licheniformis strain(s) is (are) B. licheniformis 4-2a and B. licheniformis 3-12a.

Strains B. licheniformis 3-12a, B. subtilis 4-7d, B. licheniformis 4-2a, and B. subtilis 3-5h were deposited by Danisco USA, Inc. of Waukesha, Wis. on May 13, 2011 at the Agricultural Research Service Culture Collection (NRRL), 1815 North University Street, Peoria, Ill., 61604 and given accession numbers NRRL B-50504, NRRL B-50505, NRRL B-50506, and NRRL B-50507, respectively.

The detoxifying strains, when in a viable state, are capable of detoxifying one or more mycotoxins by at least about 10-20%, 20-30%, 30-40%, 40-50%, 50-60, 60-70%, 70-80%, 80-85%, 85-90%, 90-95%, and greater than 95% as compared to a reference control (e.g., an agent with no detoxifying properties, such as a buffered saline or a strain with no detoxifying properties).

A supernatant prepared from one or more of the detoxifying strain may also be used to detoxify one or more mycotoxins. The supernatant can be diluted to any concentration that is effective for the intended purpose. The supernatant from one or more of the detoxifying strains is capable of detoxifying one or more mycotoxins by at least about 10-20%, 20-30%, 30-40%, 40-50%, 50-60, 60-70%, 60-80%, 80-85%, 85-90%, 90-95%, and greater than 95% as compared to a reference control (e.g., an agent with no detoxifying properties, such as a buffered saline or a strain with no detoxifying properties).

One or more than one detoxifying strain may be used to detoxify the mycotoxin. A detoxifying strain may be used in combination with other organism, including but not limited to other bacterial strains, and fungi. In one embodiment, one or more than one detoxifying strain is used in combination with the strain Lactobacillus brevis 1E-1 (ATCC PTA-6509)

In another embodiment, the detoxifying strains can detoxify one or more mycotoxins through biotransformation of the mycotoxin to a compound with reduced toxicity. Not to be bound by any particular theory, the detoxifying strain may detoxify one or more mycotoxins by altering the structure of the mycotoxin, for example, by post-translational modifications that reduce the toxicity of the mycotoxin. Alternatively, the detoxifying strain may detoxify one or more mycotoxins by producing a compound that counteracts the effects of the mycotoxin or competes for binding with the mycotoxin.

In another embodiment, a detoxifying strain can also be an anti-inflammatory strain.

### Anti-Inflammatory Strains

It has also been found that certain other bacterial microorganisms alleviate the gastrointestinal inflammatory response resulting from ingestion of one or more mycotoxin(s) when these microorganisms are administered to animals. These strains are referred to herein as “anti-inflammatory strain(s).”

The anti-inflammatory strains can be administered, either directly or indirectly, to animals to alleviate inflammation caused by mycotoxins. In at least some embodiments, the anti-inflammatory strains are administered with the detoxifying strains. In at least some embodiments, a strain may function as both an anti-inflammatory strain and a detoxifying strain.

In at least some embodiments, the reduction in the amount of inflammation in the gastrointestinal tract of an animal to which the strain is administered is at least one of the following: (a) by at least about 60% when the one or more mycotoxin(s) is exposed to the strain in a viable state and (b) by at least 30% when the one or more mycotoxin(s) is exposed to supernatant of the strain.

In at least some embodiments, the reduction in the amount of inflammation in the gastrointestinal tract of an animal to which the strain is administered is by at least an about 2-fold reduction in gene expression of inflammatory cytokines TNF-a and MIP-2 in intestinal cells or tissue of livestock receiving the strain(s) when the strain(s) is(are) administered to an animal.

Anti-inflammatory strains include but are not limited to lactic acid bacteria (LAB) and Bacillus strains. In at least some embodiments, the LAB strains are Lactobacillus strains such as L. acetotolerans, L. acidifarinae, L. acidipiscis, L. acidophilus, L. agilis, L. algidus, L. alimentarius, L. amylolyticus, L. amylophilus, L. amylotrophicus, L. amylovorus, L. animalis, L. antri, L. apodemi, L. aviarius, L. bifermentans, L. brevis, L. buchneri, L. camelliae, L. casei, L. catenaformis, L. ceti, L. coleohominis, L. collinoides, L. composti, L. concavus, L. coryniformis, L. crispatus, L. crustorum, L. curvatus, L. delbrueckii subsp. delbrueckii, L. delbrueckii subsp. bulgaricus, L. delbrueckii subsp. lactis, L. dextrinicus, L. diolivorans, L. equi, L. equigenerosi, L. farraginis, L. farciminis, L. fermentum, L. formicalis, L. fructivorans, L. frumenti, L. fuchuensis, L. gallinarum, L. gasseri, L. gastricus, L. ghanensis, L. graminis, L. hammesii, L. hamsteri, L. harbinensis, L. hayakitensis, L. helveticus, L. hilgardii, L. homohiochii, L. iners, L. ingluviei, L. intestinalis, L. jensenii, L. johnsonii, L. kalixensis, L. kefuranofaciens, L. kefiri, L. kimchii, L. kitasatonis, L. kunkeei, L. leichmannii, L. lindneri, L. malefermentans, L. mali, L. manihotivorans, L. mindensis, L. mucosae, L. murinus, L. nagelii, L. namurensis, L. nan ensis, L. oligofermentans, L. oris, L. panis, L. pantheris, L. parabrevis, L. parabuchneri, L. paracollinoides, L. parafarraginis, L. parakefiri, L. paralimentarius, L. paraplantarum, L. pentosus, L. perolens, L. plantarum, L. pontis, L. psittaci, L. rennini, L. reuteri, L. rhamnosus, L. rimae, L. rogosae, L. rossiae, L. ruminis, L. saerimneri, L. sakei, L. salivarius, L. sanfranciscensis, L. satsumensis, L. secaliphilus, L. sharpeae, L. siliginis, L. spicheri, L. suebicus, L. thailandensis, L. ultunensis, L. vaccinostercus, L. vaginalis, L. versmoldensis, L. vini, L. vitulinus, L. zeae, and L. zymae. In some embodiments, the Lactobacillus strain is L. brevis 1E-1 (also referred to as L. brevis strain 1E-1 or strain 1E1). Lactobacillus brevis strain 1E-1 was isolated from the intestinal tract of a healthy, weaned pig. In certain embodiments, the Lactobacillus strain is L. johnsonii PLC B6.

In certain embodiments, a LAB strains include, but are not limited to, one or more Enterococcus strain(s), such as E. avium, E. durans, E. faecalis, E. faecium, E. gallinarum, E. solitarius, etc. In some embodiments, the Enterococcus strain is Enterococcus faecium 2-1d.

Lactobacillus brevis strain 1E-1 (ATCC Accession No. PTA-6509) is available from the microorganism collection of the American Type Culture Collection, 10801 University Blvd., Manassas, Va. 20110, under accession number PTA-6509, and was deposited by Agtech Products, Inc. of Waukesha, Wis. on Jan. 12, 2005. Lactobacillus brevis strain 1E-1 was referenced in U.S. Pat. No. 7,354,757 and is publicly available from the ATCC.

Strains L. johnsonii PLC B6 and E. faecium 2-1d were deposited by Danisco USA, Inc. of Waukesha, Wis. on Jun. 3, 2011 at the Agricultural Research Service Culture Collection (NRRL), 1815 North University Street, Peoria, Ill., 61604 and given accession numbers NRRL B-50518 and NRRL B-50519, respectively.

All of the deposits were made under the provisions of the Budapest Treaty on the International Recognition of the Deposit of Microorganisms for the Purposes of Patent Procedure.

In one embodiment, the anti-inflammatory strains include Bacillus strains, including, but not limited to, B. subtilis, B. licheniformis, B. pumilus, B. coagulans, B. amyloliquefaciens, B. stearothermophilus, B. brevis, B. alkalophilus, B. clausii, B. halodurans, B. megaterium, B. circulans, B. lautus, B. thuringiensis and B. lentus strains. In at least some embodiments, the B. subtilis strain(s) is (are) B. subtilis 4-7d and B. subtilis 3-5h. In at least some embodiments, the B. licheniformis strain(s) is (are) B. licheniformis 4-2a and B. licheniformis 3-12a.

In at least some embodiments, the Bacillus strain(s) is(are) B. subtilis 3-5h (NRRL B-50507) and B. licheniformis 3-12a (NRRL B-50504). Thus, some strains described herein are both detoxifying strains and anti-inflammatory strains.

In at least some embodiments, more than one of the strain(s) described herein is/are combined to provide both a mycotoxin detoxifying effect and an anti-inflammatory effect. For example, one or more detoxifying strain(s) can be combined with one or more anti-inflammatory strain(s).

Any Bacillus, Lactobacillus or Enterococcus derivative or variant are also included and are useful in the methods described and claimed herein. In some embodiments, strains having all the characteristics of Bacillus licheniformis 3-12a, B. subtilis 4-7d, B. licheniformis 4-2a, B. subtilis 3-5h, L. brevis 1E-1, Lactobacillus johnsonii PLC B6, and Enterococcus faecium 2-1d are also included and are useful in the methods described and claimed herein.

In certain embodiments, any derivative or variant of B. licheniformis 3-12a, B. subtilis 4-7d, B. licheniformis 4-2a, B. subtilis 3-5h, L. brevis 1E-1, L. johnsonii PLC B6 and E. faecium 2-1d are also included and are useful in the methods described and claimed herein.

### Methods of Culturing Strains

The Bacillus strains are produced by fermentation of the bacterial strains. Fermentation can be started by scaling-up a seed culture. This involves repeatedly and aseptically transferring the culture to a larger and larger volume to serve as the inoculum for the fermentation, which is carried out in large stainless steel fermentors in medium containing proteins, carbohydrates, and minerals necessary for optimal growth. A non-limiting exemplary medium is TSB. After the inoculum is added to the fermentation vessel, the temperature and agitation are controlled to allow maximum growth. Once the culture reaches a maximum population density, the culture is harvested by separating the cells from the fermentation medium. This is commonly done by centrifugation.

The count of the culture can then be determined. CFU or colony forming unit is the viable cell count of a sample resulting from standard microbiological plating methods. The term is derived from the fact that a single cell when plated on appropriate medium will grow and become a viable colony in the agar medium. Since multiple cells may give rise to one visible colony, the term colony forming unit is a more useful unit measurement than cell number.

In one embodiment, each Bacillus strain is fermented between a 5×10⁸ CFU/ml level to about a 4×10⁹ CFU/ml level. In at least one embodiment, a level of 2×10⁹ CFU/ml is used. The bacteria are harvested by centrifugation, and the supernatant is removed. The supernatant can be used in the methods described herein. In at least some embodiments, the bacteria are pelleted. In at least some embodiments, the bacteria are freeze-dried. In at least some embodiments, the bacteria are mixed with a carrier. However, it is not necessary to freeze-dry the Bacillus before using them. The strains can also be used with or without preservatives, and in concentrated, unconcentrated, or diluted form.

The LAB strains can be fermented to an appropriate level. In a non-limiting example, that level is between about a 1×10⁹ CFU/ml level to about a 1×10¹⁰ CFU/ml level. The LAB strains can be grown in de Man, Rogosa and Sharpe (MRS) broth at 37° C. for 24 hours. The bacteria can be harvested by centrifugation, and the supernatant removed. The supernatant can be used in the methods described herein.

### Methods of Preparing a DFM

A composition including one or more strain(s) described herein is provided. The composition can be fed to an animal as a direct-fed microbial (DFM). One or more carrier(s) or other ingredients can be added to the DFM. The DFM may be presented in various physical forms, for example, as a top dress, as a water soluble concentrate for use as a liquid drench or to be added to a milk replacer, gelatin capsule, or gels. In one embodiment of the top dress form, freeze-dried lactic acid bacteria fermentation product is added to a carrier, such as whey, maltodextrin, sucrose, dextrose, limestone (calcium carbonate), rice hulls, yeast culture, dried starch, and/or sodium silico aluminate. In one embodiment of the water soluble concentrate for a liquid drench or milk replacer supplement, freeze-dried lactic acid bacteria fermentation product is added to a water soluble carrier, such as whey, maltodextrin, sucrose, dextrose, dried starch, sodium silico aluminate, and a liquid is added to form the drench or the supplement is added to milk or a milk replacer. In one embodiment of the gelatin capsule form, freeze-dried lactic acid bacteria fermentation product is added to a carrier, such as whey, maltodextrin, sugar, limestone (calcium carbonate), rice hulls, yeast culture dried starch, and/or sodium silico aluminate. In one embodiment, the lactic acid bacteria and carrier are enclosed in a degradable gelatin capsule. In one embodiment of the gels form, freeze-dried lactic acid fermentation product is added to a carrier, such as vegetable oil, sucrose, silicon dioxide, polysorbate 80, propylene glycol, butylated hydroxyanisole, citric acid, ethoxyquin, and/or artificial coloring to form the gel.

The strain(s) may optionally be admixed with a dry formulation of additives including but not limited to growth substrates, enzymes, sugars, carbohydrates, extracts and growth promoting micro-ingredients. The sugars could include the following: lactose; maltose; dextrose; malto-dextrin; glucose; fructose; mannose; tagatose; sorbose; raffinose; and galactose. The sugars range from 50-95%, either individually or in combination. The extracts could include yeast or dried yeast fermentation solubles ranging from 5-50%. The growth substrates could include: trypticase, ranging from 5-25%; sodium lactate, ranging from 5-30%; and, Tween 80, ranging from 1-5%. The carbohydrates could include mannitol, sorbitol, adonitol and arabitol. The carbohydrates range from 5-50% individually or in combination. The micro-ingredients could include the following: calcium carbonate, ranging from 0.5-5.0%; calcium chloride, ranging from 0.5-5.0%; dipotassium phosphate, ranging from 0.5-5.0%; calcium phosphate, ranging from 0.5-5.0%; manganese proteinate, ranging from 0.25-1.00%; and, manganese, ranging from 0.25-1.0%.

To prepare DFMs described herein, the culture(s) and carrier(s) (where used) can be added to a ribbon or paddle mixer and mixed for about 15 minutes, although the timing can be increased or decreased. The components are blended such that a uniform mixture of the cultures and carriers result. The final product is preferably a dry, flowable powder. The strain(s) can then be added to animal feed or a feed premix, added to an animal's water, or administered in other ways known in the art. A feed for an animal can be supplemented with one or more strain(s) described herein or with a composition described herein.

### Methods of Administering Strains and Compositions to an Animal

The detoxifying strains and the anti-inflammatory strains can be administered in an effective amount to animals. As used herein, the term “animal” includes but is not limited to human, mammal, amphibian, bird, reptile, pigs, cows, cattle, goats, horses, sheep, poultry, and other animals kept or raised on a farm or ranch, sheep, big-horn sheep, buffalo, antelope, oxen, donkey, mule, deer, elk, caribou, water buffalo, camel, llama, alpaca, rabbit, mouse, rat, guinea pig, hamster, ferret, dog, cat, and other pets, primate, monkey, ape, and gorilla. In some embodiments, the animals are pigs, including, but not limited to, nursery pigs, breeding stock, sows, gilts, boars, lactation-phase piglets, and finishing pigs. The strain(s) can be fed to a sow during the lactation period, although the strain(s) can be fed for different durations and at different times. In certain embodiments, the detoxifying strain(s) and/or anti-inflammatory strain(s) is/are administered to piglets by feeding the strain(s) to a gilt or sow. It is believed that the transfer to the piglets is accomplished via the fecal-oral route and/or via other routes.

This effective amount can be administered to the animal in one or more doses.

In some embodiments, the one or more Bacillus strain(s) is(are) added to an animal's feed at a rate of at least 1×10⁴ CFU/animal/day. For example, in one embodiment, the one or more Bacillus strain(s) is(are) added to pigs' feed at a rate of about 3.75×10⁵ CFU per gram of feed. It(they) can also be fed at about 1×10⁴ to about 1×10⁸ CFU/animal/day. In some embodiments, the one or more Bacillus strain(s) is(are) fed at about 1×10⁸ CFU/animal/day. In other embodiments, the one or more LAB strain(s) is(are) fed to pigs at a rate of about 1×10⁸ CFU/animal/day to about 5×10¹⁰ CFU/animal/day. It(they) can be fed at about 1×10⁹ CFU/animal/day.

For ruminants, the one or more Bacillus strain(s) is(are) fed at about 5×10⁹ CFU/hd/day. Ruminants can be fed the one or more LAB strain(s) at a rate of about 5×10¹⁰ CFU/hd/day.

For poultry, the one or more Bacillus strain(s) is(are) fed at about 1×10⁵ CFU/g feed to about 1×10¹⁰ CFU/g feed. In at least some embodiments, the one or more Bacillus strain(s) is fed at about 1×10⁵ CFU/bird/day. Poultry can be fed about 1×10⁸ CFU/bird/day. In other embodiments, the one or more LAB strain(s) is(are) fed to poultry at a rate of about 1×10⁸ CFU/animal/day to about 5×10¹⁰ CFU/animal/day. It(they) can be fed at about 1×10⁹ CFU/animal/day.

The DFM provided herein can be administered, for example, as the strain-containing culture solution, the strain-containing supernatant, or the bacterial product of a culture solution.

Administration of a DFM provided herein to an animal can increase the performance of the animal. In one embodiment, administration of a DFM provided herein to an animal can increase the average daily feed intake (ADFI), average daily gain (ADG), or feed efficiency (gain:feed; G:F) (collectively, “performance metrics”). One or more than one of these performance metrics may be improved.

The DFM may be administered to the animal in one of many ways. For example, the strain(s) can be administered in a solid form as a veterinary pharmaceutical, may be distributed in an excipient, preferably water, and directly fed to the animal, may be physically mixed with feed material in a dry form, or the strain(s) may be formed into a solution and thereafter sprayed onto feed material. The method of administration of the strain(s) to the animal is considered to be within the skill of the artisan.

When used in combination with a feed material, the feed material for ruminants can be grain or hay or silage or grass, or combinations thereof. Included amongst such feed materials are corn, dried grain, alfalfa, any feed ingredients and food or feed industry by-products as well as bio fuel industry by-products and corn meal and mixtures thereof. For monogastric diets, the feed material can include corn, soybean meal, byproducts like distillers dried grains with solubles (DDGS), and vitamin/mineral supplement. Other feed materials can also be used.

The time of administration is not crucial so long as the reductive effect on the mycotoxin's toxicity is shown. Administration is possible at any time with or without feed. However, the bacterium is preferably administered with or immediately before feed.

Thus, in at least some embodiments, the effective amount of at least one strain of bacterium is administered to an animal by supplementing a feed intended for the animal with the effective amount of at least one strain of bacterium. As used herein, “supplementing,” means the action of incorporating the effective amount of bacteria provided herein directly into the feed intended for the animal. Thus, the animal, when feeding, ingests the bacteria provided herein.

A feed for an animal comprises at least one strain of bacterium.

### Methods for Detoxifying a Mycotoxin

In one embodiment, the disclosure provides methods for detoxifying one or more mycotoxins. In one embodiment, the method for detoxifying a mycotoxin comprises administering to an animal an effective amount of a detoxifying strain or an anti-inflammatory strain or combinations thereof. In another embodiment, the strain is a Bacillus strain or a lactic acid bacteria.

In another embodiment, the method for detoxifying a mycotoxin comprises administering to an animal an effective amount of composition comprising supernatant from one or more culture(s) of one or more strain(s), wherein the strain is a lactic acid bacteria strain or a Bacillus strain.

In still another embodiment, the method for detoxifying a mycotoxin comprises administering to an animal an effective amount of a feed that is supplemented with a strain, a composition, a supernatant, or a combination of a strain and supernatant, wherein the strain or supernatant is from a lactic acid bacteria strain or a Bacillus strain.

In one embodiment, the Bacillus strain used to detoxify a mycotoxin includes but is not limited to B. licheniformis 3-12a (NRRL B-50504), B. subtilis 4-7d (NRRL B-50505), B. licheniformis 4-2a (NRRL B-50506), and B. subtilis 3-5h (NRRL B-50507), strains having all the characteristics thereof, any derivative or variant thereof, and mixtures thereof.

In still another embodiment, the lactic acid bacteria used to detoxify a mycotoxin is a Lactobacillus strain. In yet another embodiment, the lactic acid bacteria strain is L. johnsonii PLC B6 (NRRL B-50518), Enterococcus faecium 2-1d (NRRL B-50519), strains having all the characteristics thereof, any derivative or variant thereof, and mixtures thereof.

The detoxifying strains and methods disclosed herein can detoxify the mycotoxin to a percentage of the initial starting toxic properties of the mycotoxin including but not limited to 3-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, and greater than 95% as compared to a reference control (e.g., an agent with no detoxifying properties, such as a buffered saline or a strain with no detoxifying properties).

### Methods for Reducing Mold Growth

Molds are fungi that grow in the form of multicellular filaments called hyphae. A connected network of these tubular branching hyphae, called a mycelium, is considered a single organism. The dusty texture of many molds is caused by profuse numbers of asexual spores conidia formed by differentiation at the ends of hyphae. The mode of formation and shape of these spores is traditionally used to classify the mold fungi. Many of these spores are colored.

In one embodiment, the disclosure relates to methods for reducing or inhibiting mold growth. In one embodiment, the method for reducing mold growth comprises mixing a strain, a composition, supernatant or a combination of the aforementioned with an animal feed, wherein the strain is a lactic acid bacteria strain or a Bacillus strain.

In another embodiment, the method for reducing the amount of mold growth comprises supplementing an animal feed with strain, a composition, supernatant or a combination of the aforementioned, wherein the strain is a lactic acid bacteria strain or a Bacillus strain. The reduction in mold growth can be from 3-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, and greater than 95% as compared to a reference control (e.g., an agent with no mold growth reducing properties, such as a buffered saline or a strain with no growth reducing properties).

In yet another embodiment, the disclosure relates to a method for reducing mold growth comprising supplementing an animal feed with a Bacillus strain or lactic acid bacteria capable of reducing mold growth.

In another embodiment, the disclosure relates to a method for reducing mold growth comprising supplementing a cereal grain with a Bacillus strain or lactic acid bacteria capable of reducing mold growth.

In another embodiment, the methods disclosed herein are suitable for any mold for which growth control is desired including but not limited to Acremonium, Aspergillus, Cladosporium, Fusarium, Mucor, Penicillium, Rhizopus, Stachybotrys, Trichoderma, Gibberella and Alternaria.

In another embodiment, the methods disclosed herein are suitable for Acremonium strictum, Cladosporium fulvum, Fusarium graminearum, Fusarium oxysporum f.sp. cubense, Fusarium oxysporum, M. amphibiorum, M. circinelloides, M. hiemalis, M. hiemalis f. silvaticus, M. indicus, M. mucedo, M. paronychius, M. piriformis and M. racemosus.

In another embodiment, the methods disclosed herein are suitable for Penicillium aurantiogriseum; Penicillium bilaiae, Penicillium camemberti, Penicillium candidum, Penicillium chrysogenum (previously known as Penicillium notatum), Penicillium claviforme, Penicillium commune, Penicillium crustosum, Penicillium digitatum, Penicillium echinulatum, Penicillium expansum, Penicillium funiculosum, Penicillium glabrum, Penicillium glaucum, Penicillium italicum, Penicillium lacussarmientei, Penicillium marneffei, Penicillium purpurogenum, Penicillium roqueforti, Penicillium stoloniferum, Penicillium ulaiense, Penicillium verrucosum, and Penicillium viridicatum.

In another embodiment, the methods disclosed herein are suitable for Rhizopus arrhizus, Rhizopus azygosporus, Rhizopus circinans, Rhizopus microsporus, Rhizopus oligosporus, Rhizopus oryzae, Rhizopus schipperae, Rhizopus sexualis, and Rhizopus stolonifer.

In another embodiment, the methods disclosed herein are suitable for Stachybotrys albipes, Stachybotrys alternans, Stachybotrys breviuscula, Stachybotrys chartarum, Stachybotrys chlorohalonata, Stachybotrys cylindrospora, Stachybotrys dichroa, Stachybotrys elegans, Stachybotrys eucylindrospora, Stachybotrys freycinetiae, Stachybotrys kampalensis, Stachybotrys kapiti, Stachybotrys longispora, Stachybotrys mangiferae, Stachybotrys microspora, Stachybotrys nephrodes, Stachybotrys nephrospora, Stachybotrys nilagirica, Stachybotrys oenanthes, Stachybotrys parvispora, Stachybotrys ruwenzoriensis, Stachybotrys sansevieriae, Stachybotrys sinuatophora, Stachybotrys suthepensis, Stachybotrys theobromae, and Stachybotrys waitakere.

In another embodiment, the methods disclosed herein are suitable for Trichoderma aggressivum, Trichoderma amazonicum, Trichoderma asperellum, Trichoderma atroviride, Trichoderma aureoviride, Trichoderma austrokoningii, Trichoderma brevicompactum, Trichoderma candidum, Trichoderma caribbaeum var. aequatoriale, Trichoderma caribbaeum var. caribbaeum, Trichoderma catoptron, Trichoderma cremeum, Trichoderma ceramicum, Trichoderma cerinum, Trichoderma chlorosporum, Trichoderma chromospermum, Trichoderma cinnamomeum, Trichoderma citrinoviride, Trichoderma crassum, Trichoderma cremeum, Trichoderma dingleyeae, Trichoderma dorotheae, Trichoderma effusum, Trichoderma erinaceum, Trichoderma estonicum, Trichoderma fertile, Trichoderma gelatinosus, Trichoderma ghanense, Trichoderma hamatum, Trichoderma harzianum, Trichoderma helicum, Trichoderma intricatum, Trichoderma konilangbra, Trichoderma koningii, Trichoderma koningiopsis, Trichoderma longibrachiatum, Trichoderma longipile, Trichoderma minutisporum, Trichoderma oblongisporum, Trichoderma ovalisporum, Trichoderma petersenii, Trichoderma phyllostahydis, Trichoderma piluliferum, Trichoderma pleuroticola, Trichoderma pleurotum, Trichoderma polysporum, Trichoderma pseudokoningii, Trichoderma pubescens, Trichoderma reesei, Trichoderma rogersonii, Trichoderma rossicum, Trichoderma saturnisporum, Trichoderma sinensis, Trichoderma sinuosum, Trichoderma sp. MA 3642, Trichoderma sp. PPRI 3559, Trichoderma spirale, Trichoderma stramineum, Trichoderma strigosum, Trichoderma stromaticum, Trichoderma surrotundum, Trichoderma taiwanense, Trichoderma thailandicum, Trichoderma thelephoricolum, Trichoderma theobromicola, Trichoderma tomentosum, Trichoderma velutinum, Trichoderma virens, Trichoderma viride, and Trichoderma viridescens.

In another embodiment, the methods disclosed herein are suitable for Alternaria alternata, Alternaria alternantherae, Alternaria arborescens, Alternaria arbusti, Alternaria blumeae, Alternaria brassicae, Alternaria brassicicola, Alternaria burnsii, Alternaria carotiincultae, Alternaria carthami, Alternaria celosiae, Alternaria cinerariae, Alternaria citri, Alternaria conjuncta, Alternaria dauci, Alternaria dianthi, Alternaria dianthicola, Alternaria euphorbiicola, Alternaria gaisen, Alternaria helianthicola, Alternaria hungarica, Alternaria infectoria, Alternaria japonica, Alternaria limicola, Alternaria linieola, Alternaria longipes, Alternaria molesta, Alternaria panax, Alternaria perpunctulata, Alternaria petroselini, Alternaria radicina, Alternaria raphani, Alternaria saponariae, Alternaria selini, Alternaria senecionis Alternaria solani, Alternaria smyrnii, Alternaria tenuissima, Alternaria triticina, and Alternaria zinniae

In one embodiment, mold that can be inhibited by the methods disclosed herein includes but is not limited to Gibberella acerina; Gibberella acervalis; Gibberella acuminata; Gibberella africana; Gibberella agglomerata; Gibberella atrofuliginea; Gibberella atrorufa; Gibberella australis; Gibberella avenacea; Gibberella baccata; Gibberella bambusae; Gibberella bolusiellae; Gibberella bresadolae; Gibberella briosiana; Gibberella butleri; Gibberella buxi; Gibberella cantareirensis; Gibberella cicatrisata; Gibberella circinata; Gibberella coffeae; Gibberella coronicola; Gibberella creberrima; Gibberella culmicola; Gibberella cyanea; Gibberella cyanogena; Gibberella cyanospora; Gibberella cylindrospora; Gibberella effusa; Gibberella engleriana; Gibberella euonymi; Gibberella ficina; Gibberella flacca; Gibberella fujikuroi; G. fujikuroi var. subglutinans; Gibberella fusispora; Gibberella gaditjirrii; Gibberella gordonii; Gibberella gossypina; Gibberella heterochroma; Gibberella hostae; Gibberella imperatae; Gibberella indica; Gibberella intricans; Gibberella juniperi; Gibberella konza; Gibberella lagerheimii; Gibberella lateritia; Gibberella longispora; Gibberella macrolopha; Gibberella malvacearum; Gibberella mapaniae; Gibberella maxima; Gibberella nemorosa; Gibberella nygamai; Gibberella parasitica; Gibberella passiflorae; Gibberella phyllostachydicola; Gibberella polycocca; Gibberella pseudopulicaris; Gibberella pulicaris; Gibberella quinqueseptata; Gibberella rhododendricola; Gibberella rugosa; Gibberella sacchari; Gibberella spiraeae; Gibberella stilboides; Gibberella subtropics; Gibberella thapsina; Gibberella tilakii; Gibberella tricincta; Gibberella tritici; Gibberella tropicalis; Gibberella tumida; Gibberella ulicis; Gibberella venezuelana; Gibberella violacea; Gibberella vitis; Gibberella xylarioides; and Gibberella zeae.

In one embodiment, the animal feed is a cereal grain including but not limited to maize (corn), rice, wheat, barley, sorghum, millet, oats, triticale, rye, buckwheat, quinoa, and fonio.

In one embodiment, the Bacillus strain used to reduce mold growth includes but is not limited to B. licheniformis 3-12a (NRRL B-50504), B. subtilis 4-7d (NRRL B-50505), B. licheniformis 4-2a (NRRL B-50506), and B. subtilis 3-5h (NRRL B-50507), strains having all the characteristics thereof, any derivative or variant thereof, and mixtures thereof.

In still another embodiment, the lactic acid bacteria used to reduce mold growth is a Lactobacillus strain. In yet another embodiment, the lactic acid bacteria strain is L. johnsonii PLC B6 (NRRL B-50518), Enterococcus faecium 2-1d (NRRL B-50519), strains having all the characteristics thereof, any derivative or variant thereof, and mixtures thereof.

Methods of Identifying a Strain with Desired Characteristics

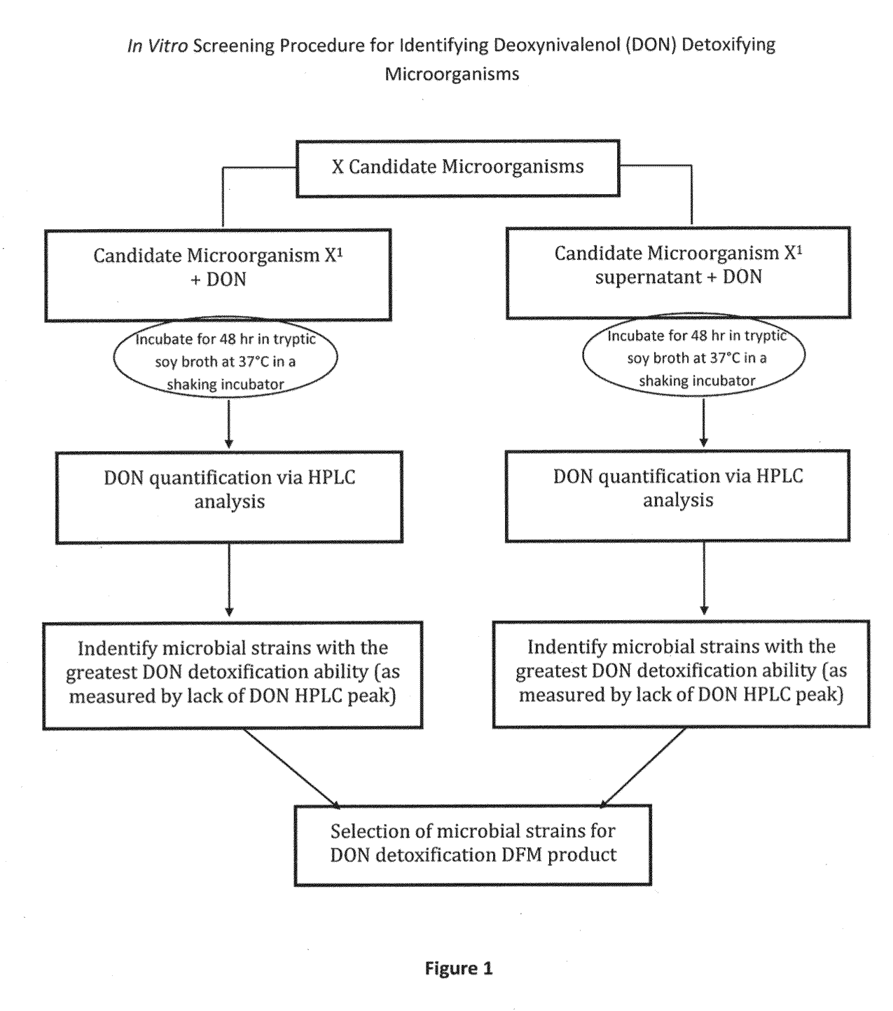

In another embodiment, the disclosure provides methods of identifying a strain that detoxifies one or more mycotoxin(s). Turning now to FIG. 1, strains that detoxify one or more mycotoxin(s) can be identified in an in vitro screening assay. This description of the screening assay will be described for DON, but it should be understood that other mycotoxins, including but not limited to T-2 and DAS, can also be screened with this assay.

A candidate microorganism, i.e., strain, is mixed with DON. Supernatant from the candidate strains is separately mixed with DON to test for the effect of the supernatant without the candidate strain. The candidate strain and the supernatant are incubated for an appropriate amount of time at an appropriate temperature under appropriate conditions. For example, as is shown in FIG. 1, the candidate strain and the supernatant from the candidate strain are incubated for 48 hours in tryptic soy broth at 37° in a shaking incubator. The amount of DON in each is quantified. In at least one embodiment, this is accomplished by HPLC. Additional candidate strains can be tested as described above. The strain(s) with a desired DON detoxification ability, such as the greatest, is determined. This can be done by selecting for a lack of a DON peak on HPLC or a reduced DON peak on HPLC. The one or more strain(s) can be further tested in an animal study such as one described below in the examples or in other studies known in the art. From either the in vitro screening, the animal study, or both, one or more strain(s) can be selected as a strain for DON detoxification.

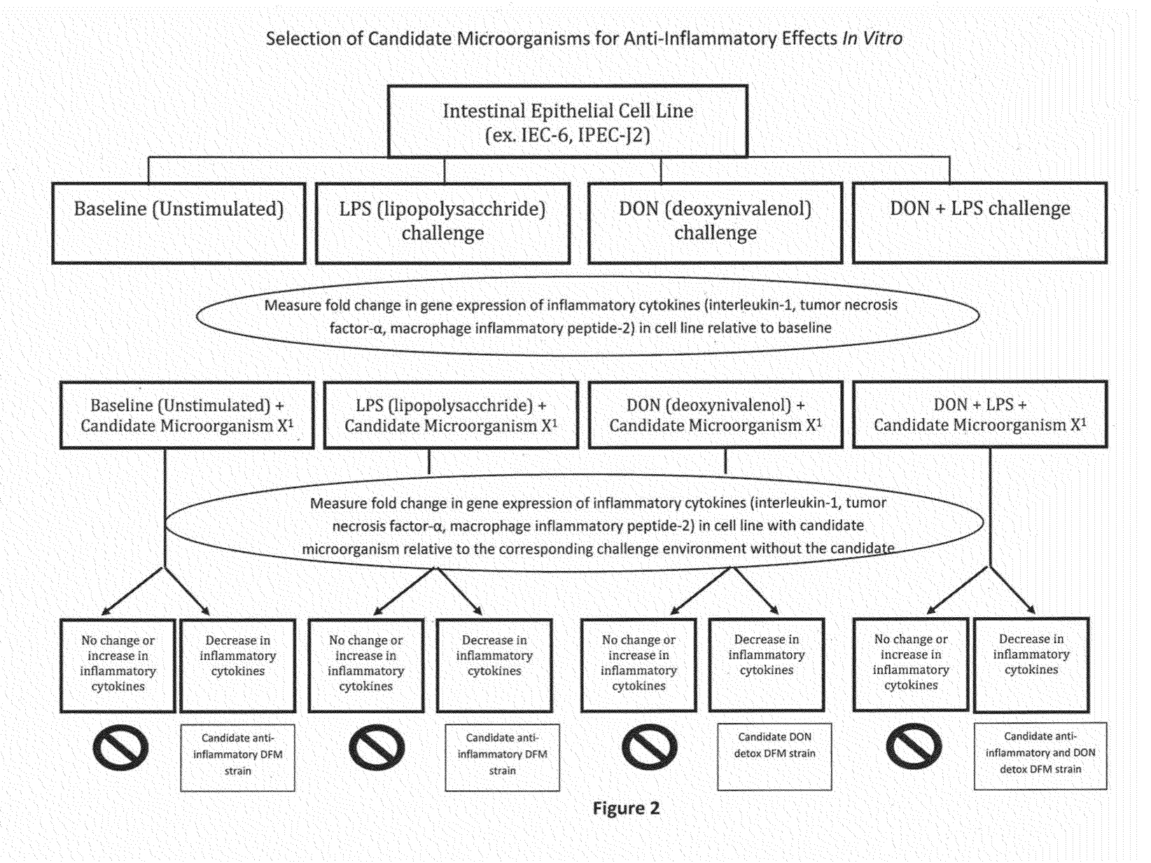

The disclosure also provides methods of identifying a strain that reduces inflammatory effects of one or more mycotoxin(s). Turning now to FIG. 2, strains that reduce inflammatory effects of mycotoxins can be identified using an in vitro screening assay. The screening assay will be described for DON, but it should be understood that other mycotoxins, including but not limited to T-2 and DAS, can also be screened with this assay. In the assay, an intestinal epithelial cell line or another cell line that has gene expression of inflammatory cytokines is used. For example, IEC-6 or IPEC-J2 can be used. The cell lines are treated as follows: 1) a baseline (unstimulated), 2) a lipopolysaccharide (LPS) challenge, 3) a DON challenge, and 4) a DON and LPS challenge. The fold change in gene expression of inflammatory cytokines is measured in the treated cell line relative to the untreated cell line, i.e. the baseline. Exemplary inflammatory cytokines that can be measured include interleukin-1 (IL-1), tumor necrosis factor-α (TNF-a), and macrophage inflammatory peptide-2 (MIP-2). However, additional inflammatory cytokines known in the art can also be used.

In addition, a second set of cell lines is used as described above except that a candidate microorganism, i.e., strain, is included. The fold change in gene expression of inflammatory cytokines is determined in the cell line with a candidate strain relative to the corresponding challenge environment, i.e., treatment, without the candidate. In at least some embodiments, the desired decrease is at least an about 2-fold reduction. Exemplary inflammatory cytokines that can be measured include those listed above. Where there is no change or an increase in inflammatory cytokines with the candidate relative to the corresponding challenge environment without the candidate, the candidate is rejected. Where there is a decrease in inflammatory cytokines with the candidate relative to the corresponding challenge environment without the candidate, the candidate remains a candidate. Further testing, such as animal trials using the candidate as a direct-fed microbial, or other testing can be performed.

Strains, compositions, and methods disclosed herein also are described by the following numbered paragraphs.

1. An isolated Bacillus strain capable of detoxifying one or more mycotoxin(s).

2. The strain of paragraph 1, wherein the detoxifying is by at least one of (a) at least about 60% when the one or more mycotoxin(s) is exposed to the strain in a viable state and (b) at least 30% when the one or more mycotoxin(s) is exposed to supernatant of the strain.

3. The strain of any of paragraphs 1-2, wherein the mycotoxin(s) is(are) selected from the group consisting of trichothecene(s), aflatoxin(s), citrinin(s), ochratoxin(s), and zearalenone(s), and combinations thereof.

4. The strain of any of paragraphs 1-3, wherein the mycotoxin(s) is(are) selected from the group consisting of T-2 toxin, diacetoxyscirpenol (DAS), fumonisin(s) and deoxynivalenol (DON), and combinations thereof.

5. The strain of any of paragraphs 1-4, wherein the mycotoxin(s) is deoxynivalenol (DON).

6. The strain of any of paragraphs 1-5, wherein the detoxification is through biotransformation of the mycotoxin(s) to a compound with reduced toxicity.

7. The strain of any of paragraphs 1-6, wherein the Bacillus strain is selected from the group consisting of the species B. subtilis and B. licheniformis, strains having all the characteristics thereof, any derivative or variant thereof, and mixtures thereof.

8. The strain of any of paragraphs 1-7, wherein the Bacillus strain is selected from the group consisting of B. licheniformis 3-12a (NRRL B-50504), B. subtilis 4-7d (NRRL B-50505), B. licheniformis 4-2a (NRRL B-50506), and B. subtilis 3-5h (NRRL B-50507), strains having all the characteristics thereof, any derivative or variant thereof, and mixtures thereof.

9. The strain of any of paragraphs 1-8, wherein the Bacillus strain is B. licheniformis 3-12a (NRRL B-50504).

10. The strain of any of paragraphs 1-8, wherein the Bacillus strain is B. subtilis 4-7d (NRRL B-50505).

11. The strain of any of paragraphs 1-8, wherein the Bacillus strain is B. licheniformis 4-2a (NRRL B-50506).

12. The strain of any of paragraphs 1-8, wherein the Bacillus strain is B. subtilis 3-5h (NRRL B-50507).

13. The strain of any of paragraphs 1-12, wherein the Bacillus strain is selected from the group consisting of B. licheniformis 3-12a (NRRL B-50504), B. subtilis 4-7d (NRRL B-50505), B. licheniformis 4-2a (NRRL B-50506), and B. subtilis 3-5h (NRRL B-50507), any derivative or variant thereof, and mixtures thereof.

14. An isolated strain capable of alleviating a gastrointestinal inflammatory response resulting from deoxynivalenol ingestion when the strain is administered to an animal, wherein the strain is a lactic acid bacteria strain or a Bacillus strain.

15. The strain of paragraph 14, wherein the strain reduces the gastrointestinal inflammatory response by at least an about 2-fold reduction in gene expression of inflammatory cytokines TNF-a and MIP-2 in intestinal cells or tissue of an animal receiving the strain when the strain is administered to an animal.

16. The strain of paragraphs 14 or 15, wherein the lactic acid bacteria strain is a Lactobacillus strain.

17. The strain of any one of paragraphs 14-16, wherein the lactic acid bacteria strain is L. johnsonii PLC B6 (NRRL B-50518), Enterococcus faecium 2-1d (NRRL B-50519), strains having all the characteristics thereof, any derivative or variant thereof, and mixtures thereof.

18. A composition comprising supernatant from one or more culture(s) of one or more strain(s) according to any one of paragraphs 1-17, and mixtures thereof.

19. A composition comprising: one or more strain(s) selected from the group consisting of Lactobacillus brevis 1E-1 (ATCC PTA-6509) and one or more strain(s) according to any of paragraphs 1-17, and mixtures thereof.

20. A feed for an animal, wherein the feed is supplemented with the isolated strain(s) according to any one of paragraphs 1-17 or with the composition(s) according to any one of paragraphs 18-19 or mixtures thereof.

21. A method for detoxifying one or more mycotoxin(s), the method comprising the step of administering to an animal an effective amount of the strain(s) according to any one of paragraphs 1-17 or with the composition(s) according to any one of paragraphs 18-19, the feed according to paragraph 20 or mixtures thereof.

22. The method of paragraph 21, wherein the mycotoxin(s) is/are trichothecene(s), aflatoxin(s), citrinin(s), ochratoxin(s), and zearalenone(s), and combinations thereof.

23. The method of paragraph 21 or 22, wherein the mycotoxin(s) is/are selected from the group consisting of T-2 toxin, diacetoxyscirpenol (DAS), fumonisin(s) and deoxynivalenol (DON) and combinations thereof.

24. The method of any one of paragraphs 21-23, wherein the mycotoxin is deoxynivalenol.

25. The method of any one of paragraphs 21-24, wherein the detoxification is through biotransformation of the mycotoxin(s) to a compound substantially without toxic characteristics.

26. A method of forming a composition, the method comprising: (a) growing, in a liquid broth, a culture including one of the isolated strain(s) according to any one of paragraphs 1-17; and (b) separating the strain from the liquid broth.

27. The method of paragraph 26, further comprising freeze drying the isolated strain and adding the freeze-dried strain to a carrier.

28. The method of paragraph 26 or 27, further comprising retaining the liquid broth after the strain has been separated from it to generate a supernatant.

29. A method of identifying a strain useful for detoxifying one or more mycotoxin(s), the method comprising:

(a) adding the mycotoxin(s) to a candidate strain to form a first mixture;

(b) adding the mycotoxin(s) to supernatant from the candidate strain to form a second mixture;

(c) incubating the first and second mixture for an appropriate amount of time at an appropriate temperature under appropriate conditions for detoxification to occur; and

(d) quantifying the amount of mycotoxin(s) in each mixture.

30. The method of paragraph 29, further comprising:

(e) repeating steps (a) through (d) for additional candidate strain(s); and

(f) selecting one or more of the candidate strain(s) that has a desired detoxification ability.

31. The method of paragraph 30, wherein the desired detoxification ability is by at least one of (a) at least about 60% when the one or more mycotoxin(s) is/are exposed to the candidate strain(s) in a viable state and (b) at least 30% when the one or more mycotoxin(s) is/are exposed to supernatant of the candidate strain(s).

32. The method of any one of paragraphs 29-31, further comprising testing the strain(s) with a desired detoxification ability in an animal study.

33. The method of any one of paragraphs 29-32, wherein the mycotoxin is deoxynivalenol.

34. A method of reducing a gastrointestinal inflammatory response resulting from ingestion of a mycotoxin, the method comprising: administering an effective amount of one or more isolated lactic acid bacteria strain(s) and/or Bacillus strain(s) capable of alleviating a gastrointestinal inflammatory response resulting from ingestion of deoxynivalenol by at least an about 2-fold reduction in gene expression of inflammatory cytokines in intestinal cells or tissue of an animal receiving the strain(s).

35. The method of paragraph 34, wherein the cytokines comprise IL-2, TNF-a, and MIP-2.

36. A method of identifying strains useful for reducing a gastrointestinal inflammatory response resulting from ingestion of a mycotoxin, the method comprising:

(a) treating a first sample with a lipopolysaccharide, a second sample with a mycotoxin, a third sample with a mycotoxin and a lipopolysaccharide, and a fourth sample with neither the lipopolysaccharide or the mycotoxin, each sample being a cell line that has gene expression of inflammatory cytokines;

(b) measuring the fold change in gene expression of inflammatory cytokines in the first, second, and third samples relative to the fourth sample;

(c) repeating steps (a) to (d) a second time with a candidate strain included with each sample;

(d) determining a fold change in gene expression of inflammatory cytokines (i) in the cell lines not including the candidate strain and (ii) in the cell lines including the candidate strain; and

(e) retaining the candidate strain as a strain useful for reducing a gastrointestinal inflammatory response resulting from ingestion of a mycotoxin when there is a decrease in the inflammatory cytokines with the candidate strain relative to corresponding treatment without the candidate strain.

37. The method of paragraph 36, wherein the cytokines comprise IL-2, TNF-a, and MIP-2.

38. The method of paragraph 36 or 37, wherein the decrease is at least an about 2-fold reduction.

39. A method comprising administering to an animal an effective amount of at least one Bacillus strain selected from the group consisting of: B. licheniformis 3-12a (NRRL B-50504), B. subtilis 4-7d (NRRL B-50505), B. licheniformis 4-2a (NRRL B-50506), and B. subtilis 3-5h (NRRL B-50507), strains having all the characteristics thereof, any derivative or variant thereof, and mixtures thereof.

40. A method comprising administering to an animal an effective amount of at least one lactic acid bacteria selected from the group consisting of L. johnsonii PLC B6 (NRRL B-50518), Enterococcus faecium 2-1d (NRRL B-50519), strains having all the characteristics thereof, any derivative or variant thereof, and mixtures thereof.

41. A method comprising administering to an animal an effective amount of at least one isolated strain capable of alleviating a gastrointestinal inflammatory response.

42. A method comprising administering to an animal an effective amount of at least one isolated strain capable of detoxifying a mycotoxin.

43. The method of paragraphs 41 or 42, wherein the strain is a Bacillus strain or a lactic acid bacteria.

44. The method of paragraphs 41-43, wherein the strain is selected from the group consisting of B. licheniformis 3-12a (NRRL B-50504), B. subtilis 4-7d (NRRL B-50505), B. licheniformis 4-2a (NRRL B-50506), and B. subtilis 3-5h (NRRL B-50507), L. johnsonii PLC B6 (NRRL B-50518), Enterococcus faecium 2-1d (NRRL B-50519), strains having all the characteristics thereof, any derivative or variant thereof, and mixtures thereof.

45. The method of paragraphs 39-44 wherein the animal is humans, primates, pigs, cows, cattle, goats, horses, sheep, poultry, dog, cat, or other house pet, and other animals kept or raised on a farm or ranch.

46. The method of paragraphs 39-45, wherein strain is administered with a carrier.

47. The method of paragraphs 39-46, wherein the animal is a pig and wherein the feeding of the strain increases performance of the pig.

48. The method of paragraphs 39-47, wherein the increase in performance comprises an increase in average daily gain.

49. A method of preparing a direct-fed microbial comprising: (a) growing, in a liquid nutrient broth, at least one Bacillus strain chosen from B. licheniformis 3-12a (NRRL B-50504), B. subtilis 4-7d (NRRL B-50505), B. licheniformis 4-2a (NRRL B-50506), and B. subtilis 3-5h (NRRL B-50507), strains having all the characteristics thereof, any derivative or variant thereof, and mixtures thereof; and (b) separating the strain from the liquid nutrient broth to from the direct-fed microbial.

50. A method of preparing a direct-fed microbial comprising: (a) growing, in a liquid nutrient broth, at least one lactic acid bacteria chosen from L. johnsonii PLC B6 (NRRL B-50518), Enterococcus faecium 2-1d (NRRL B-50519), strains having all the characteristics thereof, any derivative or variant thereof, and mixtures thereof; and (b) separating the strain from the liquid nutrient broth to from the direct-fed microbial.

51. An isolated strain of Bacillus and/or lactic acid bacteria (LAB) described in paragraphs 1-50 for use in the prevention of gastrointestinal inflammatory response.

52. The use of an isolated strain of Bacillus and/or LAB described in paragraph 1-51 in preparation of a medicament to alleviate gastrointestinal inflammatory response.

53. A feed for an animal, wherein the feed is supplemented with the isolated strain(s) selected from the group consisting of B. licheniformis 3-12a (NRRL B-50504), B. subtilis 4-7d (NRRL B-50505), B. licheniformis 4-2a (NRRL B-50506), and B. subtilis 3-5h (NRRL B-50507), L. johnsonii PLC B6 (NRRL B-50518), Enterococcus faecium 2-1d (NRRL B-50519), strains having all the characteristics thereof, any derivative or variant thereof, and mixtures thereof.

54. A feed for an animal, wherein the feed is supplemented with a supernatant from a strain selected from the group consisting of B. licheniformis 3-12a (NRRL B-50504), B. subtilis 4-7d (NRRL B-50505), B. licheniformis 4-2a (NRRL B-50506), and B. subtilis 3-5h (NRRL B-50507), L. johnsonii PLC B6 (NRRL B-50518), Enterococcus faecium 2-1d (NRRL B-50519), strains having all the characteristics thereof, any derivative or variant thereof, and mixtures thereof.

55. A feed for an animal, wherein the feed is supplemented with an isolated strain capable of alleviating a gastrointestinal inflammatory response.

56. A for an animal, wherein the feed is supplemented with an isolated strain capable of detoxifying a mycotoxin.

57. The feed of paragraphs 55 or 56, wherein the strain is a Bacillus strain or a lactic acid bacteria.

58. The feed of paragraphs 55-57, wherein the strain is selected from the group consisting of B. licheniformis 3-12a (NRRL B-50504), B. subtilis 4-7d (NRRL B-50505), B. licheniformis 4-2a (NRRL B-50506), and B. subtilis 3-5h (NRRL B-50507), L. johnsonii PLC B6 (NRRL B-50518), Enterococcus faecium 2-1d (NRRL B-50519), strains having all the characteristics thereof, any derivative or variant thereof, and mixtures thereof.

### EXAMPLES

The following Examples are provided for illustrative purposes only. The Examples are included herein solely to aid in a more complete understanding of the presently described invention. The Examples do not limit the scope of the invention described or claimed herein in any fashion.

### Example 1

### Performance Separation of Pigs Fed Deoxynivalenol-Contaminated Diets

A model to provide separation in growth performance was established using pigs in a nursery pig feeding trial fed diets formulated with a corn-based premix naturally contaminated with deoxynivalenol (DON). Prior to the start of the study, pigs were weaned into the nursery. After one week in the large nursery, pigs selected for the experiment were moved to nursery rooms with smaller pens, individually weighed, and allotted by body weight into 12 replicates. Pigs were afforded a one week adjustment period in the smaller pens prior to trial initiation, in which all pigs received the positive control diet devoid of DON. For the trial, pigs were penned individually in the smaller pens such that there were 12 individually housed pigs per treatment. Pigs were randomly assigned to one of two dietary treatments to evaluate the effect of the presence of DON in the diet on pig performance. The positive control diet consisted of a typical starter nursery diet formulated with corn devoid of DON contamination, whereas the negative control diet was formulated with DON contaminated corn at a concentration of 3 ppm total DON. Experimental diets were fed for a period of seven days, after which pigs were selected for sampling based on their performance response to vomitoxin inclusion in the feed.

Selection criteria for pigs selected for sampling were defined by feed intake and body weight gain. Previous observations led to the expectation that less than half of the pigs evaluated exhibited deleterious effects on growth performance due to DON. Other pigs fed diets formulated with DON corn exhibited growth performance similar to pigs fed the positive control diet devoid of DON. Therefore in this model, feed intake and body weight gain were determined at the end of the 7 day feeding study. Pigs were selected to represent three groups for sampling and were defined as follows:
1) pigs exhibiting a marked reduction in growth performance in response to DON as determined by depressed feed intake and body weight gain,
2) pigs exhibiting no deleterious effects on growth performance due to DON contamination in the diet, and
3) pigs representing the average of the positive control group fed diets devoid of DON.

A total of 27 pigs were sampled at the end of the trial. Twelve pigs each from the two groups of pigs fed DON diets were sampled, the two groups being those that responded negatively to DON and those that exhibited no response to DON inclusion in the diet were sampled. Three pigs from the positive control diet in which pigs were fed uncontaminated corn were also sampled for base-line comparisons to the negative control samples. The positive control pigs that were sampled exhibited average growth performance response within the positive control treatment. The average responses to feed intake and body weight gain for each of the three sampling groups are reported in Table 1. These data illustrate the responses of the pigs described as non-responders to DON ingestion that exhibit no detrimental growth performance responses, pigs described as responders to DON ingestions that exhibit marked decreases in growth performance responses and the positive control pigs that exhibited average growth performance responses when fed diets devoid of DON contamination.

TABLE 1
Average daily feed intake (ADFI), average daily gain (ADG),
and feed efficiency (gain:feed; G:F) growth responses of pigs
fed diets devoid of DON contamination (Positive Control), pigs
exhibiting depressed growth in response to DON inclusion in
the diet at 3 ppm (Responders) and pigs exhibiting no deleterious
response to 3 ppm DON inclusion (Non-Responders).

| | Positive | | Non- | | |
|---|---|---|---|---|---|
| | Control | Responders | Responders | SEM | P= |
| ADFI, kg | 0.63 | 0.42 | 0.71 | 0.05 | 0.001 |
| ADG, kg | 0.47 | 0.26 | 0.46 | 0.04 | 0.001 |
| G:F | 0.76 | 0.61 | 0.64 | 0.05 | 0.077 |

### Example 2

### Determination of Microbial Differences between Responder and Non-Responder Pigs

Tissue Processing

Gastrointestinal sections including the pars esophagus, duodenum, jejunum and ileum were collected for bacterial cell isolation. Luminal material was removed from each gut section by washing with 10 mL of sterile 0.01% sterile Peptone buffer. Tract sections were cut transversely with sterile forceps. The gut section was placed in a sterile whirl-pak bag with 99 mL of sterile peptone dilution buffer and masticated in a stomacher for 60 seconds to release colonizing or mucus associated bacteria. The masticated solution was poured into a sterile 250 mL centrifuge tube withholding the gut section. Centrifugation at 13,170×g for 10 min. was performed on the bacterial cell-containing solution. Subsequently the supernatant was discarded and 10 mL of sterile TSB+10% glycerol broth was added to the pellet, resuspended and frozen at −20° C. until subsequent DNA isolation.

DNA Isolation.

Frozen samples were thawed on ice prior to DNA isolation. Solutions were vortexed for 30 seconds to yield a heterogeneous sample. Genomic DNA was extracted from one mL of each sample using a traditional phenol-chloroform isolation procedure. The resulting genomic DNA was further purified using the High Pure PCR Template Preparation Kit (Roche Applied Science, Indianapolis, Ind.).

PCR Amplification and Terminal-Restriction Length Polymorphism (T-RFLP) Analysis.

Amplification reactions using 2 μL of purified genomic DNA from each sample were performed in triplicate to provide adequate quantity of amplified product and to reduce PCR variation. PCR amplification of a large portion of the 16S ribosomal RNA gene coding region was carried out using a 5′-tetrachloroflouresciene labeled eubacterial 16S forward primer 8F (AGAGTTTGATYMTGGCTCAG) (SEQ ID NO. 7) and the universal reverse primer 785R (ACTACCRGGGTATCTAATCC) (SEQ ID NO. 8). Reaction mixtures of 50 μL contained 1×PCR buffer, each deoxynucleoside triphosphate (dNTP) at a concentration of 280 μM, 1.5 mM MgCl₂, 12.5 pM of tetramethylammonium chloride (TMAC), 50 pM of each primer and 2.5 U of Platinum Taq (Invitrogen, Madison, Wis.). Positive and negative controls were included to monitor the effects of contaminating DNA found in commercial Taq enzymes. PCR conditions were 95° C. for 5 minutes, 30 cycles of denaturation at 95° C. for 30 seconds, annealing at 55.0° C. for 30 seconds, and extension at 72° C. for 120 seconds. The final cycle included a final extension at 72° C. for 7 minutes. Purity of PCR products was verified by running in a 1% agarose gel, staining with ethidium bromide and visualizing with an ultraviolet transilluminator. Fluorescently labeled PCR amplicons that were performed in triplicate from each sample were pooled and then purified from the primers and concentrated to 80 μL using a Qiagen PCR Clean Up Kit (Qiagen, Valencia, Calif.).

Subsequently, the cleaned sample, was split into four equal volumes. Three of the aliquots were then digested individually with 10 units of either BstUI at 60° C., BfaI, HaeIII, or MspI individually at 37° C. for 4 hours. Terminal restriction fragments (TRFs) from digestions using the restriction enzyme BstUI are denoted by the letter U followed by the size of the fragment, e.g. U100.79, while the H is used to designate TRFs from digestions using the restriction enzyme HaeIII, M for the restriction enzyme MspI, and B for the restriction enzyme BfaI. The use of three restriction enzymes improved the possibility of taxonomic identification of each TRF to the fewest number of bacterial species. DNA was analyzed using an ABI 3730xl capillary sequencer (Applied Biosystems) using GeneMapper 4.0 software (High-Throughput Sequencing and Genotyping Unit, Urbana Ill.). TRFs with sizes outside of the ranges of 20-785 basepairs and TRFs with peak heights below 50 relative fluorescence units were removed from the analysis.

Identification of Bacteria by TRF Matching.

Sample T-RFLP data from each individual sample was imported into the Bionumerics Gel Compar II package using the specialized T-RFLP extension (Applied Maths, Austin, Tex.). The Gel Compar II program was used to facilitate accurate band matching for all three restriction enzymes using a 1.0% position tolerance to define the bacterial species identified as OTUs by TRFs derived from the three restriction enzymes.

### Example 3

### Identification of Potential Deoxynivalenol (DON) Detoxifying Bacteria Based on Their Associations to Non-Responding Pigs

Bacteria were selected to have potential deoxynivalenol (DON) detoxifying capability for further screening based upon significant associations of the terminal restriction fragments (TRFs) to presence or prevalence in non-responding pigs. The forward primer yielded 7 peaks (P<0.100) from the binary data (Table 2) and 13 peaks from the quantitative data (Table 3). The reverse primer yielded 32 peaks (P<0.100) from the binary data (Table 4) and 22 peaks from the quantitative data (Table 5). Putative identifications were performed on peaks that were determined to be positively associated with non-responding animals (Table 6). Of these, peaks, those associated with Bacillus (B242.79, B551.58[rev], B245.96, and U235.89) and Lactobacillus (B268.87, B551.58[rev], H329.82[rev], H455.22[rev], and U425.85) were used as a basis for the selection of samples to plate for isolates that could have detoxification or anti-inflammatory capabilities for further screening. From these samples, a total of 830 isolates were collected. Specifically, 446 colonies were isolated from TSB for Bacillus and 384 colonies were isolated from MRS for LAB. RAPD analysis was performed to distinguish unique groups of isolates and to select strains for detoxification and anti-inflammatory immune assays.

TABLE 2
Terminal restriction fragments (TRFs) generated from the forward primer using the restriction enzymes Bfa I (B), Hae III
(H), Msp I (M), and BstU I (U) with an association to treatment when presence or absence of the TRFs are compared.

| | P-value | | | | | | | | | | | | | |
|---|---|---|---|---|---|---|---|---|---|---|---|---|---|---|
| | | Section* | Means | | | | | | | | | | | |
| TRF | Treat | Treat | E, Con | E, Res | E, Non | D, Con | D, Res | D, Non | J, Con | J, Res | J, Non | I, Con | I, Res | I, Non |
| H277.17 | 0.0838 | | 0^{AB} | 0.13^{AB} | 0^{B} | 0.67^{AB} | 0.67^{A} | 0.22^{AB} | 1^{A} | 0.75^{A} | 0.63^{AB} | 1^{A} | 0.78^{A} | 0.75^{A} |
| M75.14 | 0.0436 | | 0.33^{A} | 0.38^{A} | 0.11^{A} | 0.67^{A} | 0.67^{A} | 0.22^{A} | 0.33^{A} | 0.5^{A} | 0.25^{A} | 0.33^{A} | 0^{A} | 0^{A} |
| M220.58 | 0.0323 | 0.0035 | 0^{B} | 0^{B} | 0^{B} | 0.33^{A} | 0^{B} | 0^{B} | 0^{B} | 0^{B} | 0^{B} | 0^{B} | 0^{B} | 0^{B} |
| M535.18 | 0.0729 | | 0^{BC} | 0^{C} | 0^{C} | 0.33^{ABC} | 0^{C} | 0.11^{BC} | 0.33^{ABC} | 0.13^{BC} | 0^{C} | 1^{A} | 0.56^{AB} | 0.75^{A} |
| U249.71 | 0.0033 | | 0^{CD} | 0.25^{BCD} | 0^{D} | 1^{AB} | 0.89^{A} | 0.56^{ABC} | 1^{AB} | 1^{A} | 0.63^{ABC} | 1^{AB} | 1^{A} | 0.88^{A} |
| U235.89 | 0.0565 | | 0^{A} | 0^{A} | 0.11^{A} | 0^{A} | 0^{A} | 0^{A} | 0^{A} | 0^{A} | 0.125^{A} | 0^{A} | 0^{A} | 0.25^{A} |
| U124.03 | 0.0323 | 0.0035 | 0^{B} | 0^{B} | 0^{B} | 0.33^{A} | 0^{B} | 0^{B} | 0^{B} | 0^{B} | 0^{B} | 0^{B} | 0^{B} | 0^{B} |

^{A,B}Within a row, least squares means that do not have a common superscript letter differ, P < 0.05.

TABLE 3
Terminal restriction fragments (TRFs) generated from the forward primer using
the restriction enzymes Bfa I (B), Hae III (H), Msp I (M), and BstU I (U) with
an association to treatment when abundance of the TRFs is compared.

| | P-value | | | | | | | |
|---|---|---|---|---|---|---|---|---|
| | | Section* | Means | | | | | |
| TRF | Treat | Treat | E, Con | E, Res | E, Non | D, Con | D, Res | D, Non |
| B268.87 | | 0.0926 | 0^{B} | 0^{B} | 0^{B} | 71.67^{B} | 0^{B} | 0^{B} |
| B245.96 | 0.0164 | | 423.67^{BC} | 797.25^{C} | 295.44^{C} | 2087^{BC} | 575.56^{C} | 1002.67^{C} |
| B242.79 | | 0.0629 | 0^{AB} | 0^{B} | 0^{B} | 391.33^{A} | 23.67^{AB} | 0^{B} |
| H263.87 | 0.0005 | 0.0325 | 0^{CD} | 0^{D} | 86.44^{D} | 2624.33^{BCD} | 299.56^{CD} | 1172.33^{CD} |
| H222.63 | 0.0202 | | 0^{A} | 0^{A} | 0^{A} | 233^{A} | 0^{A} | 0^{A} |
| M178.16 | 0.006 | 0.0602 | 0^{B} | 0^{B} | 0^{B} | 3033.67^{AB} | 1520.22^{B} | 877.33^{B} |
| M220.58 | 0.0323 | 0.0035 | 0^{B} | 0^{B} | 0^{B} | 127^{A} | 0^{B} | 0^{B} |
| M535.18 | 0.003 | 0.0266 | 0^{BC} | 0^{C} | 0^{C} | 316.67^{BC} | 0^{C} | 78.78^{BC} |
| M558.75 | 0.0154 | | 0^{BC} | 0^{C} | 0^{C} | 191.67^{BC} | 0^{C} | 29.22^{BC} |
| U425.85 | | 0.0838 | 0^{AB} | 0^{B} | 0^{B} | 153.67^{AB} | 37.44^{AB} | 448.44^{A} |
| U246.71 | 0.0681 | | 0^{AB} | 350.34^{B} | 0^{B} | 4105^{AB} | 2576^{AB} | 1287.67^{AB} |
| U124.03 | 0.0323 | 0.0035 | 0^{B} | 0^{B} | 0^{B} | 109.67^{A} | 0^{B} | 0^{B} |
| U110 | 0.0023 | 0.0317 | 1254.33^{BC} | 1771.5^{BC} | 897.89^{C} | 3957^{BC} | 1226^{C} | 1976.22^{BC} |

| | Means | | | | | |
|---|---|---|---|---|---|---|
| TRF | J, Con | J, Res | J, Non | I, Con | I, Res | I, Non |
| B268.87 | 0^{B} | 94.25^{B} | 81^{B} | 682^{A} | 217^{B} | 239.63^{AB} |
| B245.96 | 2549.67^{BC} | 874.63^{C} | 2138.63^{BC} | 9169^{A} | 3353.22^{BC} | 5127.75^{AB} |
| B242.79 | 0^{AB} | 0^{B} | 125.25^{AB} | 65.67^{AB} | 0^{B} | 18.25^{AB} |
| H263.87 | 2681.33^{BCD} | 850.38^{CD} | 1061.25^{CD} | 10426^{A} | 3400.33^{BC} | 5506.63^{B} |
| H222.63 | 199^{A} | 85.75^{A} | 23.25^{A} | 101.67^{A} | 118^{A} | 23.13^{A} |
| M178.16 | 6731.67^{A} | 3074.25^{AB} | 898.25^{B} | 2692.33^{AB} | 1959^{B} | 2115.88^{B} |
| M220.58 | 0^{B} | 0^{B} | 0^{B} | 0^{B} | 0^{B} | 0^{B} |
| M535.18 | 196.67^{BC} | 58.63^{BC} | 0^{C} | 1558^{A} | 425.22^{BC} | 589.625^{B} |
| M558.75 | 206^{BC} | 44.63^{BC} | 82.5^{BC} | 999^{A} | 412.67^{B} | 414.13^{B} |
| U425.85 | 79.33^{AB} | 330.25^{AB} | 134.88^{AB} | 274^{AB} | 368.89^{AB} | 378.75^{AB} |
| U246.71 | 7846^{A} | 4222.25^{AB} | 2348.88^{AB} | 3553.33^{AB} | 3561^{AB} | 2909.75^{AB} |
| U124.03 | 0^{B} | 0^{B} | 0^{B} | 0^{B} | 0^{B} | 0^{B} |
| U110 | 3567^{BC} | 1441.5^{C} | 1913.13^{BC} | 13540.67^{A} | 4301.33^{BC} | 6355.63^{B} |

^{A,B,C,D}Within a row, least squares means that do not have a common superscript letter differ, P < 0.05.

TABLE 4
Terminal restriction fragments (TRFs) generated from the reverse primer using the restriction enzymes Bfa I (B), Hae
III (H), Msp I (M), and BstU I (U) with an association to treatment when presence or absence of the TRFs is compared.

| | | Section* | | | | | | | | | | | | |
|---|---|---|---|---|---|---|---|---|---|---|---|---|---|---|
| TRF | Treat | Treat | E, Con | E, Res | E, Non | D, Con | D, Res | D, Non | J, Con | J, Res | J, Non | I, Con | 1, Res | I, Non |
| B560.74 | 0.0369 | | 0.33^{A} | 0.75^{A} | 0.67^{A} | 0.33^{A} | 0.78^{A} | 0.56^{A} | 0.33^{A} | 0.38^{A} | 0.5^{A} | 0^{A} | 0.78^{A} | 0.25^{A} |
| B551.58 | 0.0171 | | 0.33^{A} | 0.63^{A} | 0.78^{A} | 0.67^{A} | 0.78^{A} | 0.89^{A} | 0.33^{A} | 0.75^{A} | 0.88^{A} | 0.33^{A} | 0.78^{A} | 0.88^{A} |
| B400.27 | | 0.035 | 0.67^{A} | 1^{A} | 0.44^{A} | 0.33^{A} | 0.56^{A} | 0.78^{A} | 1^{A} | 0.38^{A} | 0.38^{A} | 0^{A} | 0.44^{A} | 0.5^{A} |
| B187.70 | 0.0323 | 0.0036 | 0^{B} | 0^{B} | 0^{B} | 0^{B} | 0^{B} | 0^{B} | 0.33^{A} | 0^{B} | 0^{B} | 0^{B} | 0^{B} | 0^{B} |
| B149.18 | 0.0585 | | 0.33^{A} | 0.25^{A} | 0^{A} | 0.67^{A} | 0.22^{A} | 0.11^{A} | 0^{A} | 0^{A} | 0^{A} | 0.33^{A} | 0.11^{A} | 0.13^{A} |
| B98.55 | 0.0963 | | 0^{AB} | 0^{B} | 0^{B} | 0.67^{AB} | 0.22^{AB} | 0.11^{AB} | 1^{A} | 0.63^{AB} | 0.38^{AB} | 0.67^{AB} | 0.56^{AB} | 0.63^{AB} |
| B96.75 | 0.0879 | | 0^{A} | 0^{A} | 0^{A} | 0^{A} | 0^{A} | 0^{A} | 0^{A} | 0.38^{A} | 0.13^{A} | 0^{A} | 0.22^{A} | 0^{A} |
| B50.75 | 0.0323 | 0.0035 | 0^{B} | 0^{B} | 0^{B} | 0.33^{A} | 0^{B} | 0^{B} | 0^{B} | 0^{B} | 0^{B} | 0^{B} | 0^{B} | 0^{B} |
| U771.42 | 0.0283 | | 0^{A} | 0.13^{A} | 0^{A} | 0^{A} | 0^{A} | 0^{A} | 0^{A} | 0.13^{A} | 0^{A} | 0^{A} | 0.33^{A} | 0^{A} |
| U691.96 | 0.0667 | | 0^{A} | 0^{A} | 0.11^{A} | 0^{A} | 0^{A} | 0.11^{A} | 0^{A} | 0^{A} | 0.25^{A} | 0^{A} | 0^{A} | 0^{A} |
| U284.34 | 0.0218 | | 0^{A} | 0.63^{A} | 0.33^{A} | 0^{A} | 0.33^{A} | 0.11^{A} | 0^{A} | 0.13^{A} | 0^{A} | 0.33^{A} | 0.33^{A} | 0^{A} |
| U277.58 | 0.05 | | 0.33^{A} | 0.88^{A} | 0.78^{A} | 0.33^{A} | 0.89^{A} | 0.44^{A} | 0.33^{A} | 0.75^{A} | 0.88^{A} | 1^{A} | 0.89^{A} | 0.75^{A} |
| U275.83 | | 0.0947 | 1^{A} | 0.88^{A} | 1^{A} | 0.67^{A} | 1^{A} | 1^{A} | 1^{A} | 1^{A} | 0.88^{A} | 1^{A} | 1^{A} | 1^{A} |
| U273.94 | 0.0323 | 0.0035 | 1^{A} | 1^{A} | 1^{A} | 0.67^{B} | 1^{A} | 1^{A} | 1^{A} | 1^{A} | 1^{A} | 1^{A} | 1^{A} | 1^{A} |
| U247.01 | 0.0304 | | 0^{AB} | 0^{B} | 0^{B} | 0.33^{AB} | 0.56^{AB} | 0.22^{AB} | 1^{A} | 0.88^{A} | 0.38^{AB} | 1^{A} | 0.56^{AB} | 0.5^{AB} |
| U207.03 | 0.058 | | 0.33^{A} | 0.13^{A} | 0.22^{A} | 0.67^{A} | 0.33^{A} | 0.11^{A} | 0.67^{A} | 0.5^{A} | 0.38^{A} | 0.67^{A} | 0.22^{A} | 0.13^{A} |
| U201.06 | 0.0751 | 0.0923 | 0.33^{AB} | 0^{B} | 0^{B} | 0^{AB} | 0.11^{AB} | 0^{B} | 0^{AB} | 0.13^{AB} | 0.13^{AB} | 0.67^{A} | 0.22^{AB} | 0^{B} |
| U152.99 | 0.0323 | 0.0036 | 0^{B} | 0^{B} | 0^{B} | 0^{B} | 0^{B} | 0^{B} | 0^{B} | 0^{B} | 0^{B} | 0.33^{A} | 0^{B} | 0^{B} |
| U126.05 | | 0.0705 | 0^{A} | 0^{A} | 0.11^{A} | 0^{A} | 0^{A} | 0^{A} | 0^{A} | 0^{A} | 0^{A} | 0.33^{A} | 0^{A} | 0^{A} |
| U73.01 | | 0.0563 | 0^{A} | 0^{A} | 0^{A} | 0^{A} | 0^{A} | 0^{A} | 0^{A} | 0.13^{A} | 0^{A} | 0.33^{A} | 0^{A} | 0^{A} |
| H672.82 | | 0.0587 | 0^{A} | 0^{A} | 0^{A} | 0^{A} | 0^{A} | 0^{A} | 0^{A} | 0^{A} | 0.13^{A} | 0.33^{A} | 0^{A} | 0^{A} |
| H476.98 | 0.038 | 0.0482 | 0^{B} | 0.75^{AB} | 0.78^{AB} | 0.33^{AB} | 0.89^{AB} | 1^{A} | 1^{AB} | 0.89^{AB} | 0.63^{AB} | 0.67^{AB} | 0.89^{AB} | 0.63^{AB} |
| H430.68 | | 0.0853 | 0^{A} | 0^{A} | 0^{A} | 0.33^{A} | 0^{A} | 0.22^{A} | 0.33^{A} | 0.5^{A} | 0.13^{A} | 0.67^{A} | 0.22^{A} | 0^{A} |
| H384.30 | 0.0969 | | 0^{A} | 0.38^{A} | 0.11^{A} | 0.67^{A} | 0.33^{A} | 0.22^{A} | 0.67^{A} | 0.38^{A} | 0.25^{A} | 1^{A} | 0.33^{A} | 0.38^{A} |
| H351.86 | | 0.0577 | 0.33^{A} | 0.13^{A} | 0^{A} | 0.67^{A} | 0.22^{A} | 0.22^{A} | 0.33^{A} | 0.38^{A} | 0.25^{A} | 0.67^{A} | 0.22^{A} | 0.13^{A} |
| H329.82 | 0.097 | | 1^{A} | 0.5^{A} | 0.66^{A} | 1^{A} | 0.78^{A} | 0.89^{A} | 1^{A} | 0.88^{A} | 0.88^{A} | 1^{A} | 0.78^{A} | 1^{A} |
| H279.36 | | 0.0863 | 0^{A} | 0^{A} | 0^{A} | 0^{A} | 0^{A} | 0^{A} | 0^{A} | 0.25^{A} | 0^{A} | 0.33^{A} | 0^{A} | 0.13^{A} |
| H276.94 | 0.0369 | | 0^{ABC} | 0^{BC} | 0^{C} | 0.33^{ABC} | 0.33^{ABC} | 0.11^{ABC} | 0.67^{ABC} | 0.75^{A} | 0.25^{ABC} | 1^{A} | 0.67^{AB} | 0.5^{ABC} |
| H271.91 | 0.0327 | | 0^{A} | 0^{A} | 0^{A} | 0^{A} | 0^{A} | 0.22^{A} | 0^{A} | 0^{A} | 0.25^{A} | 0.33^{A} | 0^{A} | 0.25^{A} |
| H153.65 | 0.054 | | 0^{A} | 0^{A} | 0^{A} | 0.33^{A} | 0^{A} | 0.11^{A} | 0.67^{A} | 0.13^{A} | 0.25^{A} | 0.33^{A} | 0.22^{A} | 0^{A} |
| H75.16 | | 0.0706 | 0^{A} | 0^{A} | 0^{A} | 0^{A} | 0^{A} | 0^{A} | 0.33^{A} | 0^{A} | 0^{A} | 0^{A} | 0.33^{A} | 0^{A} |
| H57.58 | 0.0927 | | 0^{A} | 0^{A} | 0^{A} | 0^{A} | 0^{A} | 0.11^{A} | 0^{A} | 0.13^{A} | 0.38^{A} | 0^{A} | 0.22^{A} | 0.38^{A} |

^{A,B,C}Within a row, least squares means that do not have a common superscript letter differ, P < 0.05.

TABLE 5
Terminal restriction fragments (TRFs) generated from the forward primer using
the restriction enzymes Bfa I (B), Hae III (H), Msp I (M), and BstU I (U) with
an association to treatment when abundance of the TRFs is compared.

| | | Section* | | | | | | |
|---|---|---|---|---|---|---|---|---|
| TRF | Treat | Treat | E, Con | E, Res | E, Non | D, Con | D, Res | D, Non |
| B560.74 | 0.0429 | | 24.33^{A} | 151.5^{A} | 59.22^{A} | 17.33^{A} | 752.44^{A} | 90.22^{A} |
| B400.27 | | 0.0272 | 44^{A} | 64.5^{A} | 26.56^{A} | 17.33^{A} | 37.33^{A} | 44.67^{A} |
| B187.70 | 0.0323 | 0.0036 | 0^{B} | 0^{B} | 0^{B} | 0^{B} | 0^{B} | 0^{B} |
| B96.75 | 0.0816 | | 0^{A} | 0^{A} | 0^{A} | 0^{A} | 0^{A} | 0^{A} |
| B50.75 | 0.0323 | 0.0035 | 0^{B} | 0^{B} | 0^{B} | 25^{A} | 0^{B} | 0^{B} |
| U771.42 | 0.0306 | | 0^{A} | 6.38^{A} | 0^{A} | 0^{A} | 0^{A} | 0^{A} |
| U691.96 | 0.0673 | | 0^{A} | 0^{A} | 6.11^{A} | 0^{A} | 0^{A} | 5.78^{A} |
| U404.48 | 0.0184 | 0.0227 | 0^{B} | 0^{B} | 0^{B} | 88^{AB} | 12.78^{B} | 0^{B} |
| U284.34 | 0.0181 | | 0^{A} | 40^{A} | 18.56^{A} | 0^{A} | 20.11^{A} | 6.67^{A} |
| U266.37 | 0.091 | | 49.33^{A} | 29.63^{A} | 95.89^{A} | 245.67^{A} | 818^{A} | 146.56^{A} |
| U152.99 | 0.0323 | 0.0036 | 0^{B} | 0^{B} | 0 | 0^{B} | 0^{B} | 0^{B} |
| U126.05 | | 0.0962 | 0^{A} | 0^{A} | 6.22^{A} | 0^{A} | 0^{A} | 0^{A} |
| H672.82 | | 0.0682 | 0^{A} | 0^{A} | 0^{A} | 0^{A} | 0^{A} | 0^{A} |
| H455.22 | 0.07 | 0.0925 | 27.67^{B} | 103.5^{B} | 58.78^{B} | 347^{B} | 206^{B} | 261.56^{B} |
| H430.68 | 0.0719 | 0.06 | 0^{A} | 0^{A} | 0^{A} | 32^{A} | 0^{A} | 15.11^{A} |
| H362.44 | 0.027 | 0.001 | 0^{B} | 55^{B} | 11.78^{B} | 17.33^{B} | 67^{B} | 35^{B} |
| H351.86 | 0.0095 | | 18^{A} | 12.5^{A} | 0^{A} | 57.67^{A} | 15.33^{A} | 13.78^{A} |
| H271.91 | 0.0326 | | 0^{A} | 0^{A} | 0^{A} | 0^{A} | 0^{A} | 16.44^{A} |
| H259.48 | | 0.0727 | 0^{B} | 0^{B} | 0^{B} | 0^{B} | 5.78^{B} | 0^{B} |
| H224.40 | | 0.0103 | 0^{B} | 7.88^{B} | 0^{B} | 55.33^{A} | 0^{B} | 0^{B} |
| H153.65 | 0.0106 | | 0^{A} | 0^{A} | 0^{A} | 38^{A} | 0^{A} | 6.78^{A} |
| H75.16 | | | 0^{A} | 0^{A} | 0^{A} | 0^{A} | 0^{A} | 0^{A} |

| TRF | J, Con | J, Res | J, Non | I, Con | I, Res | I, Non |
|---|---|---|---|---|---|---|
| B560.74 | 22.67^{A} | 774.5^{A} | 180.25^{A} | 13.38^{A} | 613.89^{A} | 0^{A} |
| B400.27 | 67^{A} | 21^{A} | 26^{A} | 0^{A} | 30.22^{A} | 31.38^{A} |
| B187.70 | 0^{B} | 0^{B} | 0^{B} | 17^{A} | 0^{B} | 0^{B} |
| B96.75 | 0^{A} | 23^{A} | 0^{A} | 0^{A} | 17.67^{A} | 6.75^{A} |
| B50.75 | 0^{B} | 0^{B} | 0^{B} | 0^{B} | 0^{B} | 0^{B} |
| U771.42 | 0^{A} | 8.88^{A} | 0^{A} | 0^{A} | 19.44^{A} | 0^{A} |
| U691.96 | 0^{A} | 0^{A} | 14.5^{A} | 0^{A} | 0^{A} | 0^{A} |
| U404.48 | 0^{B} | 32.25^{B} | 21.38^{B} | 285.33^{A} | 75.78^{B} | 34.25^{B} |
| U284.34 | 0^{A} | 19.75^{A} | 0^{A} | 21.33^{A} | 23.11^{A} | 0^{A} |
| U266.37 | 0^{A} | 704.75^{A} | 181.25^{A} | 1291.67^{A} | 978.33^{A} | 370.13^{A} |
| U152.99 | 0^{B} | 0^{B} | 0^{B} | 16.67^{A} | 0^{B} | 0^{B} |
| U126.05 | 0^{A} | 0^{A} | 0^{A} | 16.67^{A} | 0^{A} | 0^{A} |
| H672.82 | 0^{A} | 0^{A} | 6.88^{A} | 17.33^{A} | 0^{A} | 0^{A} |
| H455.22 | 614^{B} | 284.88^{B} | 278.13^{B} | 3518^{A} | 915.44^{B} | 1269.38^{B} |
| H430.68 | 18.33^{A} | 33.5^{A} | 6.25^{A} | 43.33^{A} | 12.11^{A} | 0^{A} |
| H362.44 | 330.67^{A} | 28.88^{B} | 31^{B} | 66.67^{B} | 28.11^{B} | 14.13^{B} |
| H351.86 | 32.33^{A} | 19.63^{A} | 13.25^{A} | 54^{A} | 16^{A} | 6.88^{A} |
| H271.91 | 0^{A} | 0^{A} | 13.75^{A} | 17^{A} | 0^{A} | 14.25^{A} |
| H259.48 | 0^{B} | 0^{B} | 6.25^{B} | 42^{A} | 12.44^{AB} | 6.88^{B} |
| H224.40 | 0^{B} | 6.5^{B} | 0^{B} | 0^{B} | 0^{B} | 6.38^{B} |
| H153.65 | 48^{A} | 6.25^{A} | 21.5^{A} | 32^{A} | 11.22^{A} | 0^{A} |
| H75.16 | 17^{A} | 0^{A} | 0^{A} | 0^{A} | 5.56^{A} | 0^{A} |

^{A,B}Within a row, least squares means that do not have a common superscript letter differ, P < 0.05.

TABLE 6
Putative Identification of TRFs generated using the restriction
enzymes Bfa I (B), Hae III (H), Msp I (M), and BstU I (U). All
peaks are from the forward primer unless noted with [rev].

| Peaks | Putative ID |
|---|---|
| B 242.79 | Bacillus |
| | Clostridium |
| | E. coli |
| | Salmonella |
| B 268.87 | Lactobacillus |
| B 551.58(rev) | Bacteroidetes |
| | Prevotella |
| | Bacillus |
| | Lactobacillus |
| B245.96 | Bacillus |
| | Clostridium |
| | Streptococcus |
| | Lactococcus |
| H 329.82(rev) | Lactobacillus |
| H153.65(rev) | Bacteroidetes |
| H263.87 | Bacteroides |
| | Prevotella |
| H271.91(rev) | Clostridium |
| H455.22(rev) | Clostridium |
| | Lactobacillus |
| | Enterococcus |
| H57.58(rev) | ? |
| U 110 | Bacteroidetes |
| | Streptococcus |
| | Lactococcus |
| U235.89 | Bacillus |
| U425.85 | Lactobacillus |
| U691.96(rev) | ? |

### Example 4

### In Vitro Screening of Identified Candidate Bacteria for Deoxynivalenol (DON) Detoxification Ability

The 446 Bacillus strains that were isolated based on the presence of TRFs associated with the Non-Responder pig group were screened for their ability to biotransform deoxynivalenol (DON) to less toxic compounds in vitro. Deoxynivalenol was added to TSB at 3 ppm, inoculated with 1% of a 24 hour culture, and then incubated for 48 hours at 37° C. with shaking. For the supernatant tests, the strains were grown in the presence of DON, cells were pelleted, and the supernatant was filtered through a 0.2 micron filter. An additional 3 ppm DON was then added to the supernatant and incubated at 37° C. for 24 hours.

One milliliter of each sample was passed through the DONtest immunoaffinity column (Vicam, Watertown, Mass.) at the rate of 1 drop per second until the column was dry. The column was then washed with 5.0 mLs of water at a rate of 1 drop per second until the column was dried. The deoxynivalenol was then eluted off of the DONtest immunoaffinity column with 1.0 mL of HPLC grade methanol into a glass vial. The methanol was allowed to evaporate in a fume hood until the sample was completely dry. Each sample was then resuspended in 500 μl of 10% acetonitrile in water.

Each sample was injected (700) onto a reverse phase, Synergi 4 μm, Hydro-RP, 250×4.6 mm Phenomenex HPLC column with precolumn (Phenomenex Inc., Torrance, Calif.) at 30° C. The mobile phase was 10% acetonitrile in water at flow rate of 1.2 mL/minute and each sample was run for 10 minutes. Peaks were detected using a photo diode array at 220 nm.

The first round of detoxification screening yielded 10 strains that had detoxification of greater than 80% (Table 7). In the second round of detoxification screening, the supernatant was filtered from the bacterial cells after each strain was incubated for 24 hours in media containing 3 ppm DON. The supernatant was prepared by sterile filtering 24 hour cell suspension in TSB that had been inoculated with 1% of each strain after having been grown in the presence of 3 ppm DON. The supernatant of strain 4-2a had the highest detoxification of 37.77%, with 4 other strains' supernatants also detoxifying DON at greater than 30% (Table 8).

TABLE 7
The percent detoxification of DON by each strain
at forty eight hours. All Bacillus strains were
inoculated into media containing 3 ppm DON.

| Strain | Area | Concentration | % reduction |
|---|---|---|---|
| 3-1g | 188587 | 1.94 | 35.3 |
| 3-2a | 188185 | 1.93 | 35.6 |
| 3-2h | 309021 | 3.00 | 00.0 |
| 3-3a | 148327 | 1.14 | 62.1 |
| 3-3c | 142250 | 1.01 | 66.2 |
| 3-5h | 58583 | 0.00 | 100.0 |
| 3-11a | 86015 | 0.00 | 100.0 |
| 3-12a | 172046 | 1.61 | 46.3 |
| 3-12e | 99633 | 0.16 | 94.6 |
| 4-1b | 43896 | 0.00 | 100.0 |
| 4-2a | 95709 | 0.08 | 97.2 |
| 4-4h | 73838 | 0.00 | 100.0 |
| 4-5e | 106853 | 0.31 | 89.8 |
| 4-6c | 76604 | 0.00 | 100.0 |
| 4-6e | 63396 | 0.00 | 100.0 |
| 4-7d | 58258 | 0.00 | 100.0 |

TABLE 8
The percent detoxification of DON by the supernatant of each Bacillus
strain after 24 hours. All strains were inoculated into media containing
3 ppm DON. The supernatant was prepared by sterile filtering 24 hr
cell suspension in TSB that had been inoculated with 1% of each strain
after having been grown in the presence of 3 ppm DON.

| | % Detoxification |
|---|---|
| Strain | Supernatant |
| 3-1g | 24.45 |
| 3-2a | 6.83 |
| 3-2h | 4.71 |
| 3-3c | 9.62 |
| 3-3a | 33.61 |
| 3-5h | 11.94 |
| 3-4a | 12.94 |
| 3-12a | 17.15 |
| 3-11a | 25.28 |
| 4-1b | 24.53 |
| 3-12e | 9.65 |
| 4-4h | 38.46 |
| 4-2a | 37.77 |
| 4-6c | 25.58 |
| 4-5e | 35.24 |
| 4-6e | 30.80 |
| 4-7d | 20.70 |

### Example 5

### In Vitro Screening of Identified Candidate Bacteria for Anti-Inflammatory Effects to Alleviate Inflammation Resulting from Deoxynivalenol (DON) in the Porcine IPEC-J2 Cell Line

The porcine intestinal epithelial cell line IPEC-J2 was used to determine the inflammatory response to deoxynivalenol (DON) and screen bacterial direct-fed microbial (DFM) candidates for the ability to alleviate this inflammatory response. A total of 96 lactic acid bacteria and Bacillus strains were screened in the cell culture assay to determine changes in inflammatory cytokine gene expression responses to DON and bacterial candidates. IPEC-J2 cells were incubated alone (unstimulated), with lipopolysaccharide (LPS), with DON, with LPS+DON, with candidate DFM, with LPS+DFM, with DON+DFM, and with LPS+DON+DFM. The plate template design is illustrated in FIG. 3. IPEC-J2 cells were grown to confluence and plated in 24-well tissue culture plates with Dulbecco's Modified Eagle's Medium (DMEM)/Ham's F12 medium (Invitrogen, Carlsbad, Calif.) containing 5% FBS (Atlanta Biologicals, Lawrenceville, Ga.) and 0.1% antibiotic-antimycotic (Atlanta Biologicals). Once confluent, IPEC-J2 cells were washed three times with antibiotic free DMEM and were then incubated for 1 hour at 37° C. with media alone or with 10 ng/mL LPS. After the incubation, media was removed and DFM and 10 ng/mL DON were added to appropriate wells and incubated for 1 hour at 37° C. After the incubation, cells were washed twice with antibiotic free DMEM and were incubated in 380 μL TRIzol for 5 minutes. Samples were removed from plates, placed in 2 mL microcentrifuge tubes, snap frozen, and stored at −80° C. until RNA isolation. To separate RNA from the organic phase, 2 ml heavy phase lock gel tubes were used (Five Prime, Inc., Gaithersburg, Md.). RNA cleanup was done using the RNeasy mini kit (Qiagen, Inc., Valencia, Calif.) and DNase digestion was done using the RNase-Free DNase kit (Qiagen, Inc.). cDNA was synthesized using the qScript cDNA SuperMix (VWR, Radnor, Pa.) immediately following the RNA isolation. Real-time PCR was used to determine gene expression of the IPEC-J2 cells using primer sets displayed in Table 9. β-actin was used as a reference gene.

Table 10 displays the IPEC-J2 porcine intestinal epithelial cell gene expression response to LPS, DON and a selection of the candidate DFM strains. DON alone and in the presence of LPS increased the gene expression of TNF-α by about 5-fold. Strains PIG d1 and PIG e1 both decreased the gene expression of the inflammatory cytokine TNF-α 3 to 4-fold, although they did not have much effect in alleviating the inflammatory response resulting from DON. Strains PIC d6 and PID c1 both alleviated most of the increase in TNF-α gene expression associated with DON in the IPEC-J2 cell line, whereas strain 2-1d prevented the increase in gene expression of TNF-α resulting from the administration of DON and LPS together.

TABLE 9
Porcine primer sets for cell culture
screening using the IPEC-J2 cell line.

| | | PCR |
|---|---|---|
| Primer | | Product |
| Name | Primer Sequence | (bp) |
| IL-6 | F: 5′-GCCACCTCAGACAAAATGCT-3′ | 143 |
| | (SEQ ID NO. 9) | |
| | R: 5′-TCTGCCAGTACCTCCTTGCT-3′ | |
| | (SEQ ID NO. 10) | |
| IL-8 | F: 5′-ACTTCCAAACTGGCTGTTGCC-3′ | 307 |
| | (SEQ ID NO. 11) | |
| | R: 5′-CTGCTGTTGTTGTTGCTTCTC-3′ | |
| | (SEQ ID NO. 12) | |
| TNF-α | F: 5′-CCCAAGGACTCAGATCATCG-3′ | 102 |
| | (SEQ ID NO. 13) | |
| | R: 5′-ATACCCCACTCTGCCATTGGA-3′ | |
| | (SEQ ID NO. 14) | |
| β-actin | F: 5′-GGACCTGACCGACTACCTCA-3′ | 115 |
| | (SEQ ID NO. 15) | |
| | R: 5′-GCGACGTAGCAGAGCTTCTC-3′ | |
| | (SEQ ID NO. 16) | |

TABLE 10
TNF-α expression of IPEC-J2 cells in the presence
of the candidate DFM strain alone, the strain and DON,
the strain and LPS, or the strain and LPS/DON combination¹

| | Treatment | Control | 2-1d | PLC B6 | PID c1 | PIG d1 | PIG e1 |
|---|---|---|---|---|---|---|---|
| IL-6 | Unstimulated | 0.15 | −0.72 | −2.41 | −3.87 | −1.73 | −3.64 |
| | DON² | −0.86 | −2.44 | −2.04 | −2.64 | −1.97 | −3.07 |
| | LPS³ | 0.29 | −0.83 | −2.74 | −1.95 | −1.93 | −2.95 |
| | LPS/DON⁴ | −0.48 | −2.50 | −2.86 | −2.23 | −4.54 | −3.56 |
| IL-8 | Unstimulated | 0.43 | −1.06 | −1.66 | −3.25 | −0.75 | −2.06 |
| | DON² | 0.56 | −1.59 | −1.45 | −1.81 | −0.03 | −0.71 |
| | LPS³ | 0.88 | −0.54 | −1.92 | 0.55 | −1.79 | −1.40 |
| | LPS/DON⁴ | 1.31 | −1.82 | −1.68 | −0.26 | −1.82 | −1.58 |
| TNF-α | Unstimulated | −0.13 | −2.03 | −0.94 | −1.66 | −3.44 | −4.29 |
| | DON² | 4.43 | 2.81 | 1.58 | 1.91 | 3.12 | 2.82 |
| | LPS³ | 0.16 | −0.53 | −0.21 | −0.55 | −3.96 | −2.00 |
| | LPS/DON⁴ | 5.72 | 0.55 | 1.70 | 3.16 | 2.51 | 3.36 |

¹Fold changes displayed are relative to unstimulated IPEC-J2 cells.
²Fold changes of IPEC-J2 cells with DON alone.
³Fold changes of IPEC-J2 cells with LPS alone.
⁴Fold changes of IPEC-J2 cells with LPS/DON alone.

### Example 6

### In Vitro Screening of Identified Candidate Bacteria for Anti-Inflammatory Effects to Alleviate Inflammation Resulting from Deoxynivalenol (DON) in the RAT IEC-6 Cell Line

The same bacterial direct-fed microbial candidate strains from Example 5 were further screened using the IEC-6 rat intestinal epithelial cell line. Plate set up and study design was the same as in Example 5 with the exception that the IEC-6 cell line was used in place of the IPEC-J2 cell line and primers specific for rat genes associated with inflammatory cytokines were used for gene expression assays (Table 11).

Table 12 displays five candidate DFM strains from the IEC-6 cell line screening. Strains 3-5h and 3-12a are Bacillus strains. Strains 1E1, PLC B6, and 2-1d are lactic acid bacteria Lactobacillus strains. Deoxynivalenol (DON), lipopolysaccharide (LPS), and the LPS/DON combination resulted in an inflammatory response as indicated by an upregulation of TNF-α and MIP-2 expression relative to unstimulated cells. Reduced expression of the inflammatory cytokines TNF-α and MIP-2 resulted after administration of DON and LPS in combination with all five strains individually.

TABLE 11
Rat primer sets used in Round 3 DON
Microbial Screening-Immunology IEC-6

| | | PCR |
|---|---|---|
| Primer | | Product |
| Name | Primer Sequence | (bp) |
| TNF-α | F: 5′-GGCAGCCTTGTCCCTTGAAGAG-3′ | 171 |
| | (SEQ ID NO. 17) | |
| | R: 5′-GTAGCCCACGTCGTAGCAAACC-3′ | |
| | (SEQ ID NO. 18) | |
| MIP-2 | F: 5′-GCAAGGCTAACTGACCTGGA-3′ | 64 |
| | (SEQ ID NO. 19) | |
| | R: 5′-CTTTGATTCTGCCCGTTGAG-3′ | |
| | (SEQ ID NO. 20) | |
| β-actin | F: 5′-TGACGAGGCCCAGAGCAAGA-3′ | 331 |
| | (SEQ ID NO. 21) | |
| | R: 5′-ATGGGCACAGTGTGGGTGAC-3′ | |
| | (SEQ ID NO. 22) | |

TABLE 12
TNF-α and MIP-2 expression of IEC-6 cells in the presence
of the MYCO candidate DFM strain alone, the strain and DON, the
strain and LPS, or the strain and LPS/DON combination using qPCR¹

| | Treatment | Control | 3-5h | 3-12a | 1E1 | PIC b6 | 2-1d |
|---|---|---|---|---|---|---|---|
| TNF-α | Unstimulated | 0.04 | 12.45 | 19.30 | 0.97 | 0.73 | −0.31 |
| | DON² | 8.99 | 51.87 | 113.66 | 9.78 | 4.04 | 12.18 |
| | LPS³ | 35.83 | 24.29 | 17.32 | 17.32 | 18.64 | 13.28 |
| | LPS/DON⁴ | 387.71 | 224.47 | 166.64 | 219.52 | 163.58 | 288.63 |
| MIP-2 | Unstimulated | 0.07 | 9.11 | 27.02 | −0.61 | −1.47 | −0.58 |
| | DON² | 9.25 | 43.53 | 150.45 | 6.12 | 4.62 | 6.20 |
| | LPS³ | 39.42 | 23.81 | 82.12 | 22.10 | 26.71 | 14.45 |
| | LPS/DON⁴ | 419.23 | 153.65 | 165.98 | 169.95 | 173.90 | 260.13 |

¹Fold changes displayed are relative to unstimulated IEC-6 cells.
²Fold changes of IEC-6 cells with DON alone.
³Fold changes of IEC-6 cells with LPS alone.
⁴Fold changes of IEC-6 cells with LPS/DON alone.

### Example 7

### Selection of Direct-Fed Microbial Strains that Alone or in Combination will Detoxify the Deoxynivalenol (DON) Mycotoxin and Alleviate Intestinal Inflammation Associated with DON Ingestion in Feed

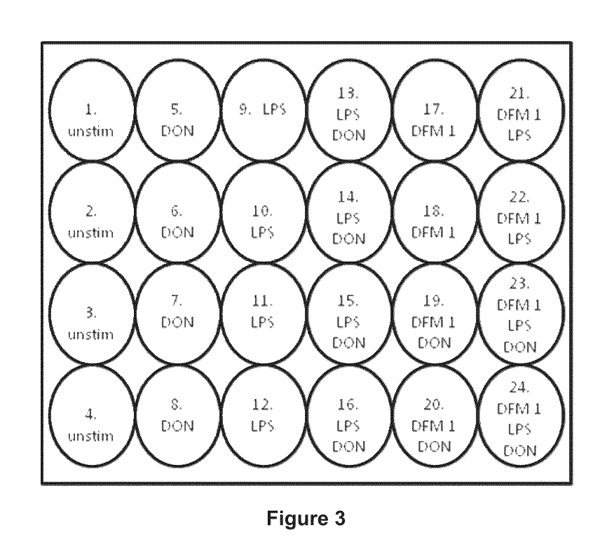

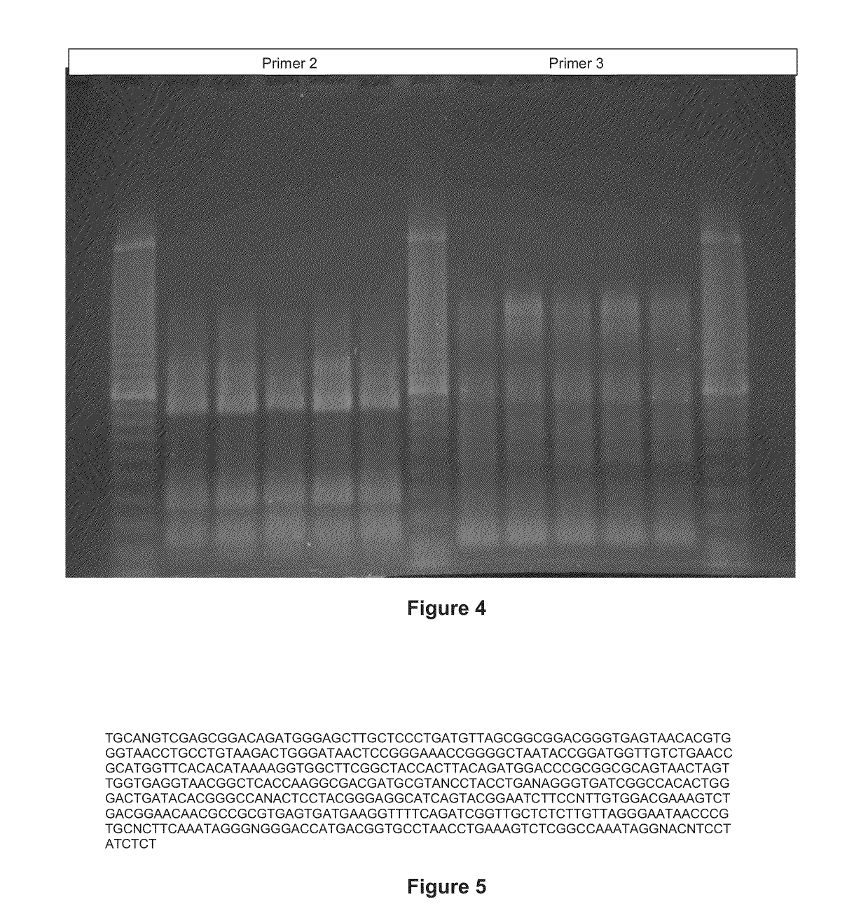

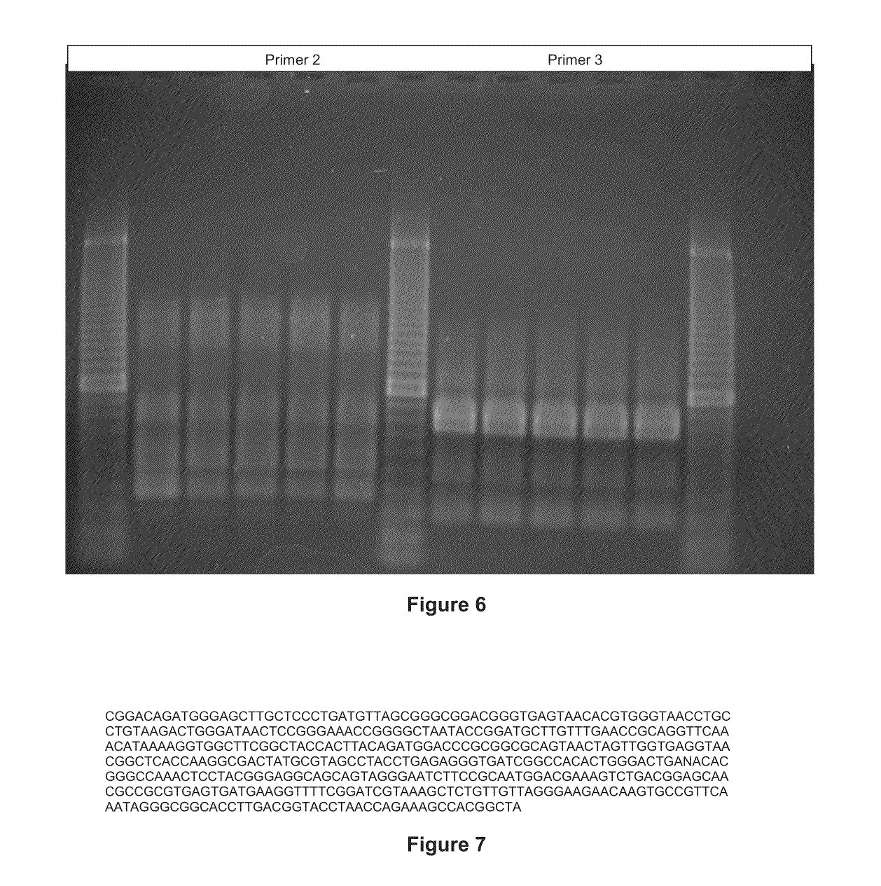

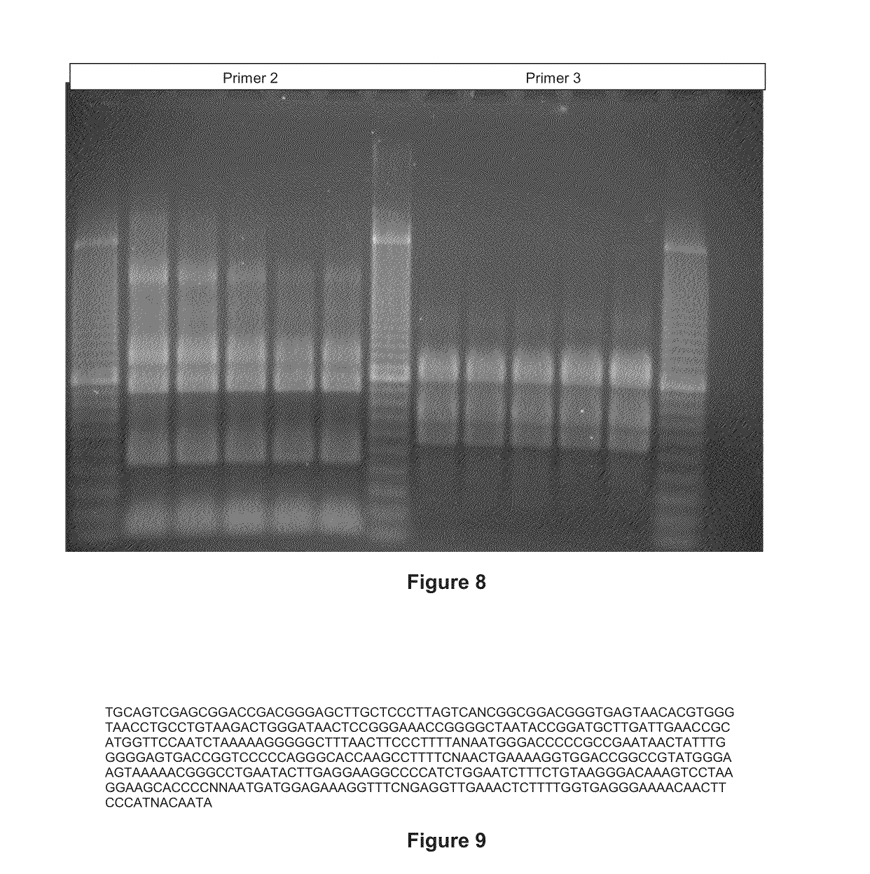

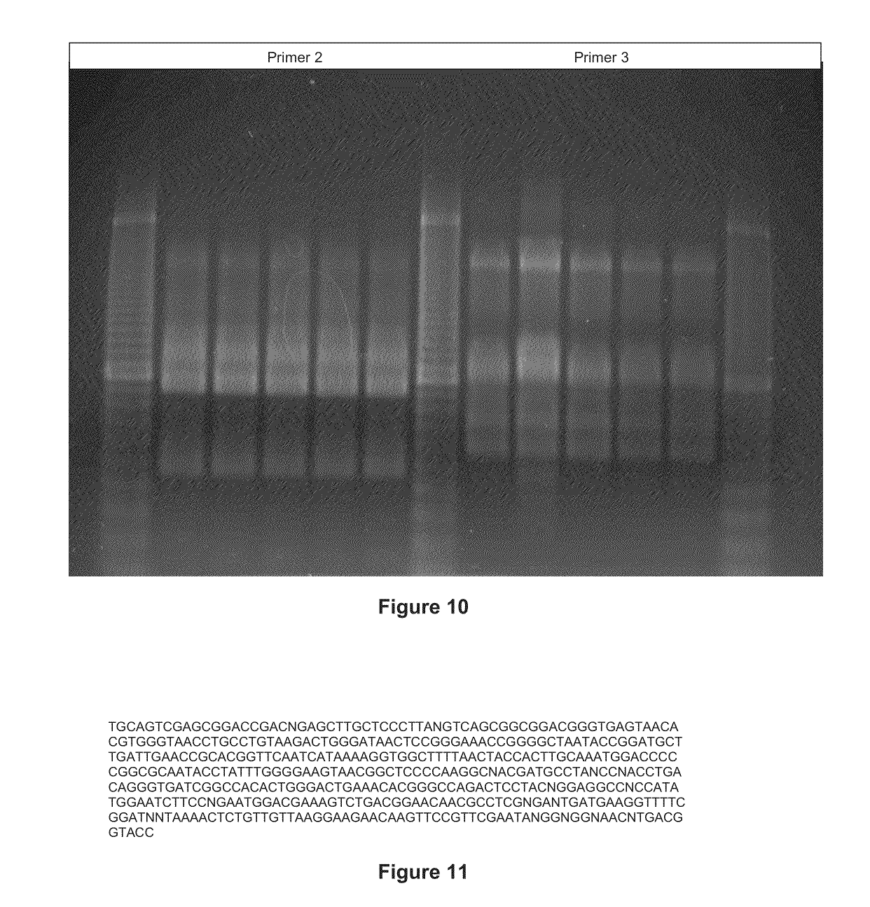

Based on detoxification ability and the ability to elicit an anti-inflammatory immune response in the presence of deoxynivalenol (DON), four additional isolates were chosen as candidates for one or more direct-fed microbial(s) (DFM(s)). RAPD profiles and partial 16S rDNA sequences of each strain were determined. The four strains are: Bacillus subtilis 4-7d (FIGS. 4 & 5), Bacillus subtilis 3-5h (FIGS. 6 & 7), Bacillus licheniformis 4-2a (FIGS. 8 & 9) and Bacillus licheniformis 3-12a (FIGS. 10 & 11). Lactobacillus brevis strain 1E-1 is also a candidate for one or more direct-fed microbial(s) (DFM(s)).

### Example 8

### Animal Testing of Direct-Fed Microbial Strains

Following selection of strains based upon detected differences in microbial populations between pigs considered negative responders to vomitoxin (VOM), i.e., deoxynivalenol (DON) in the diet compared to pigs that exhibit no detrimental effects on performance when exposed to vomitoxin, and detoxification and anti-inflammatory immunomodulation screening, animal feeding trials will be conducted to validate the efficacy of the selected strains in swine production conditions. In brief, weanling pigs will be administered a positive control diet formulated with uncontaminated corn, a negative control diet formulated with vomitoxin contaminated corn, and the negative control diet supplemented with candidate direct-fed microbial (DFM) product strains in various combinations or singly as warranted by data gleaned for each strain.

It is expected that at minimum, there will be an improvement of about 15% in animal body weight gain, feed intake, and feed efficiency when the DFM is administered in feed contaminated with 3 ppm VOM compared to animals fed the 3 ppm VOM diet devoid of the DFM.

### Example 9

### Use of Bacteria to Inhibit DON-Producing Mold

Novel microbial strain technology was assessed in a screening assay to determine the bioefficacy of four Bacillus strains and two strains of lactic acid bacteria as mold inhibitors. Bacillus strains were Bacillus subtilis strains 4-7d and 3-5, and Bacillus licheniformis strains 4-2a and 3-12a. Lactic acid bacteria strains were Lactobacillus johnsonii PLCB6 and Enterococcus faecium 2-1d. Wheat samples naturally contaminated with 6.0 ppm deoxynivalenol (DON) were plated on malachite green agar (MGA; Alborch et al., 2010). Selective culture media for the detection of Fusarium infection in conventional and transgenic maize kernels (Letters in Applied Microbiology. 50: 270-275) was used to selectively grow Fusarium and other related species of mold. Plates were incubated at 25° C. for 5 days. Isolated colonies were picked into potato dextrose broth (PDB; BD Difco, Franklin Lakes, N.J.) and grown at 25° C. for 3 days with shaking at a rate of 200 rpm.

DNA was isolated from 0.5 mL of the culture using the UltraClean Microbial DNA Isolation Kit (MoBio Laboratories, Carlsbad, Calif.) following the Experienced User Protocol in the Instruction Manual version: 05172010. The remainder of the culture was centrifuged at 5,000 g for 10 minutes and resuspended in 5.0 mL of PDB containing 20% glycerol. Polymerase chain reaction (PCR) (Henry, et al., 2000) was used for the identification of Aspergillus using internal transcribed spacer regions 1 and 2 (Journal of Clinical Microbiology. 38(4): 1510-1515). PCR was performed on the intergenic transcribed spacer region (ITS) to identify the species of each of the resulting mold isolates. Three isolates were determined to be Gibberella, one isolate was each found to be Fusarium, Penicillium, and Cladosporium (Table 13).

TABLE 13
Mold isolates used in the inhibition assay.

| Mold | Source | ITS Sequence ID | Accession | Max Ident |
|---|---|---|---|---|
| 1 | Wheat with 6 ppm DON | Gibberella moniliformis | GU257903.1 | 99% |
| 2 | Wheat with 6 ppm DON | Penicillium brevicompactum | HM210834.1 | 99% |
| 3 | Wheat with 6 ppm DON | Fusarium proliferatum | GQ924905.1 | 99% |
| 4 | Wheat with 6 ppm DON | Gibberella moniliformis | HQ637284.1 | 99% |
| 5 | Wheat with 6 ppm DON | Cladosporium colombiae | JQ346204.1 | 99% |
| 6 | Wheat with 6 ppm DON | Gibberella moniliformis | HQ637284.1 | 99% |
| 7 | ATCC 10911 | Fusarium oxysporum | JN020659.1 | 99% |

To determine mold inhibition by bacterial strains, MGA plates were prepared by surface plating 100 μL of previously prepared stock of six environmental mold isolates and one ATCC mold isolate, all identified in Table 13. Plates were incubated at 25° C. for 4 days to allow a lawn of mold growth to form. Bacillus subtilis strains 4-7d and 3-5h, as well as Bacillus licheniformis strains 4-2a and 3-12a were grown in TSB (BD Difco, Franklin Lakes, N.J.) at 37° C. for 24 hours. Lactic acid bacteria (LAB) isolates Lactobacillus johnsonii PLCB6 and Entrococcus faecium 2-1d were grown in de Man, Rogosa and Sharpe (MRS; BD Difco, Franklin Lakes, N.J.) broth at 37° C. for 24 hours.

Cells from all six bacterial cultures were pelleted by centrifugation at 5,000 g for 10 minutes. The resulting supernatant was filtered through a 0.2 micron filter and then spotted in triplicate onto MGA plates containing the mold lawns and incubated at 25° C. for 24 hours. After incubation, zones of inhibition in the mold lawns were measured in triplicate and recorded in millimeters of diameter. Data was analyzed using Proc Mixed procedure of SAS (v. 9.1.3, SAS Institute, Inc., Cary, N.C.) statistical software comparing control (application of bacteriocin free media) with candidate strain bacteriocin treatment (significance level α=0.05, means were separated using Tukey HSD). All bacterial strains tested resulted in inhibition activity against at least one of the seven mold isolates screened (Table 14). Bacillus subtilis 3-5h and B. licheniformis 4-2a each inhibited (P<0.001) one mold isolate. B. subtilis 4-7d inhibited (P<0.001) three of the mold isolates. B. licheniformis 3-12a and L. johnsonii PLC B6 each inhibited (P<0.001) four of the mold isolates. E. faecium 2-1d inhibited (P<0.001) six of the mold isolates.

TABLE 14
Average zone of clearance in mm averaged over 3 replicate plates. *

| Strain | Mold 1. | Mold 2. | Mold 3. | Mold 4. | Mold 5. | Mold 6. | Mold 7. |
|---|---|---|---|---|---|---|---|
| BS3-5h | 0.0^{a} | 0.0^{a} | 0.0^{a} | 0.0^{a} | 0.0^{a} | 0.0^{a} | 5.3^{c} |
| BL3-12a | 6.0^{bc} | 9.3^{b} | 0.0^{a} | 0.0^{a} | 5.3^{b} | 0.0^{a} | 3.3^{b} |
| BL4-2a | 5.7^{bc} | 0.0^{a} | 0.0^{a} | 0.0^{a} | 0.0^{a} | 0.0^{a} | 0.0^{a} |
| BS4-7d | 4.0^{b} | 0.0^{a} | 0.0^{a} | 6.7^{c} | 5.0^{b} | 0.0^{a} | 0.0^{a} |
| PLC B6 | 7.3^{bc} | 0.0^{a} | 3.3^{b} | 3.0^{b} | 6.7^{b} | 0.0^{a} | 0.0^{a} |
| 2-1d | 9.0^{c} | 0.0^{a} | 4.0^{b} | 4.0^{b} | 8.7^{c} | 11.3^{b} | 5.7^{c} |
| Control | 0.0^{a} | 0.0^{a} | 0.0^{a} | 0.0^{a} | 0.0^{a} | 0.0^{a} | 0.0^{a} |
| P-Value | <0.001 | <0.001 | <0.001 | <0.001 | <0.001 | <0.001 | <0.001 |
| SEM | 0.8 | 0.7 | 0.4 | 0.6 | 0.8 | 0.9 | 0.6 |

* differing superscripts within a column indicate significant difference from control at P ≦ 0.05, separation of means by Tukey HSD.

### Example 10

### The Use of Bacillus Direct-Fed Microbials to Alleviate the Negative Growth Performance Effects in Pigs Fed Deoxynivalenol Contaminated Diets

Bacillus direct-fed microbial (DFM) strains BS3-5h (NRRL-B 50507, Strain A) and BS4-7d (NRRL-B 50505, Strain B) were tested in a commercial nursery study for efficacy to improve growth performance responses in pigs fed grain-based diets naturally contaminated with deoxynivalenol (DON). In preparation for the feeding trial, piglets were weaned and fed a standard commercial wheat based diet for 7 days to acclimatize. After acclimatization, piglets were fed a wheat based diet for 6 days containing 2.5 ppm of deoxynivalenol (DON) derived from a wheat source naturally contaminated with DON. During the responder phase when DON contaminated diets were fed, 135 DON sensitive animals showing depressed growth and feed intake were selected for the feeding trial. Following the responder phase, there was a 7 day re-acclimatization phase on standard commercial wheat based diet before Bacillus DFM product was tested.

During the main feeding trial to assess strain efficacy, two control diets and three treatment diets were tested. The positive control (PC) diet did not contain DON or DFM, the negative control (NC) diet contained 2.5 ppm DON via inclusion of naturally contaminated wheat as before, but no DFM. Treatment diets contained 2.5 ppm DON and a total amount of 1.6×10⁸ CFU DFM per gram of diet with varying relative amounts of strains A and B as illustrated in Table 15.

TABLE 15
Dietary treatment and relative strain inclusion.*

| | Bacillus subtilis | Bacillus subtilis |
|---|---|---|
| | BS3-5h | BS4-7d |
| Dietary treatment | Strain A | Strain B |
| Positive control | | |
| (PC) | | |
| Negative control | | |
| (NC) | | |
| A | 100% | |
| B | | 100% |
| A + B | 50% | 50% |

*Strain A = Bacillus subtilis BS3-5h (NRRL-B 50507); strain B = Bacillus subtilis BS4-7d (NRRL-B 50505).

Pig performance response was monitored by measuring average daily gain (ADG), average daily feed intake (ADFI) and feed conversion rate (Feed to Gain, F:G) over the 14 day duration of the trial. Data were analyzed using Proc Mixed procedure of SAS (v. 9.1.3, SAS Institute, Inc., Cary, N.C.) with a significance level α=0.10. Data were normalized for 231b initial body weight. Separation of treatment means was performed using Tukey HSD.

There was a clear separation between PC and NC in both phases of the trial, with generally more variability of animal performance in the unchallenged PC compared to NC and treatment animals (Table 16). Animals on NC diet had lower (P<0.05) ADG and ADFI compared with PC animals, but F:G between the positive and negative control treatments was not affected.

During the first week of treatment, ADG and ADFI of pigs fed any of the three treatment diets did not differ from pigs fed the negative control diet, and responses were lower (P<0.05) than pigs fed the positive control diet devoid of DON. During the second week of the trial, ADG of pigs fed strain combination A+B did not differ from pigs fed the negative control diet or the positive control diet devoid of DON, indicating this strain combination partially alleviated the detrimental response on body weight gain resulting from inclusion of the DON toxin in the diet. Similar to first trial week, F:G was not affected during the second week of the trial.

To further determine the implication for DON detoxification efficacy of each strain tested in wheat-based diets naturally contaminated with the DON mycotoxin, data were analyzed as 2×2 factorial ANOVA using Proc Mixed procedure of SAS (v. 9.1.3, SAS Institute, Inc., Cary, N.C.) with a significance level α=0.10 and data normalized for 231b initial body weight. Factors were Bacillus DFM Strain A (Bacillus subtilis 3-5h, NRRL-B 50507) and Strain B (Bacillus subtilis 4-7d, NRRL-B 50505), either present or absent in the experimental wheat based diets with 2.5 ppm DON. The inclusion of B. subtilis 3-5h (Strain A) in the DON-contaminated diet resulted in less (P=0.10) feed required per unit of body weight gain compared to pigs fed the same diet devoid of this DFM strain (Table 17). The inclusion of B. subtilis 4-7d (Strain B) in the DON-contaminated diet resulted in greater (P=0.06) ADG and ADFI compared to pigs fed the same diet devoid of Strain B.

These data indicate that the inclusion, both singly and combined, of Strain A (Bacillus subtilis 3-5h, NRRL-B 50507) and Strain B (Bacillus subtilis 4-7d, NRRL-B 50505) in diets that are naturally contaminated with DON reduces the detrimental effects on animal growth resulting from mycotoxin contamination.

TABLE 16
Animal performance over 14 day grower pig study.¹

| Performance | Positive | Negative | Tested Strain(s)⁴ | | | |
|---|---|---|---|---|---|---|
| Measure | control (PC)² | control (NC)³ | A | B | A + B | P-Value |

Week 1

| | | | | | | |
|---|---|---|---|---|---|---|
| ADG (lb) | 1.040 ± 0.351^{c} | 0.722 ± 0.060^{ab} | 0.729 ± 0.060^{ab} | 0.696 ± 0 058^{ab} | 0.734 ± 0.063^{ab} | <0.001 |
| ADFI (lb) | 1.567 ± 0.381^{b} | 1.141 ± 0.053^{a} | 1.220 ± 0.053^{a} | 1.166 ± 0.052^{a} | 1.150 ± 0.054^{a} | <0.001 |
| F:G (lb:lb) | 1.525 ± 0.169 | 1.646 ± 0.224 | 1.991 ± 0.222^{b} | 1.758 ± 0.217 | 1.356 ± 0.226 | 0.73 |

Week 2

| | | | | | | |
|---|---|---|---|---|---|---|
| ADG (lb) | 1.459 ± 0.324^{c} | 1.178 ± 0.067^{ab} | 1.237 ± 0.066^{ab} | 1.202 ± 0.067^{ab} | 1.255 ± 0.068^{bc} | 0.02 |
| ADFI (lb) | 2.048 ± 0.519^{c} | 1.623 ± 0.074^{ab} | 1.703 ± 0.073^{ab} | 1.737 ± 0.071^{b} | 1.712 ± 0.075^{ab} | <0.001 |
| F:G (lb:lb) | 1.432 ± 0.220 | 1.392 ± 0.044 | 1.366 ± 0.043 | 1.478 ± 0.044 | 1.378 ± 0.044 | 0.47 |

¹Data analysis normalized for start weight of 23.0 lb; n = 15 animals per treatment; ADG, Average Daily Gain; ADFI, Average Daily Feed Intake; F:G, Feed to Gain; data presented in averages ± standard error;
^{a,b,c}superscripts indicate significant difference of means for P ≦ 0.05, separation of means by Tukey HSD;
²Positive control diet without deoxynivalenol (DON);
³Negative Control diet with DON at 2.5 ppm;
⁴Negative control diet supplemented with Bacillus based direct-fed microbial at 1.6 × 10⁸ CFU DFM per gram of diet with differing relative amounts of strain A = Bacillus subtilis BS3-5h (NRRL-B 50507) and strain B = Bacillus subtilis BS4-7d (NRRL-B 50505) as illustrated in Table 15

TABLE 17
Main effect response of two Bacillus strains tested in DON
contaminated wheat-based diets on pig growth performance. ¹

| Factor specific analysis | | | | | |
|---|---|---|---|---|---|
| Bacillus subtilis BS3- | | | Bacillus subtilis BS4- | | |
| 5h Strain A | | | 7d Strain B | | |
| | | P- | | | P- |
| 0 | + | value | 0 | + | value |

Week 1

| | | | | | | |
|---|---|---|---|---|---|---|
| ADG (lb) | 0.692 | 0.722 | 0.54 | 0.726 | 0.688 | 0.41 |
| ADFI (lb) | 1.159 | 1.184 | 0.61 | 1.169 | 1.175 | 0.91 |
| F:G (lb:lb) | 1.672 | 1.690 | 0.91 | 1.747 | 1.615 | 0.40 |

Week 2

| | | | | | | |
|---|---|---|---|---|---|---|
| ADG (lb) | 1.198 | 1.207 | 0.87 | 1.149 | 1.255 | 0.06 |
| ADFI (lb) | 1.685 | 1.677 | 0.90 | 1.616 | 1.747 | 0.06 |
| F:G (lb:lb) | 1.427 | 1.377 | 0.10 | 1.403 | 1.402 | 0.98 |

¹ Factorial data analysis normalized for start weight of 23.0 lb; n = 15 animals per treatment; ADG, Average Daily Gain; ADFI, Average Daily Feed Intake; F:G, Feed to Gain; Strain A = Bacillus subtilis BS3-5h (NRRL-B 50507); strain B = Bacillus subtilis BS4-7d (NRRL-B 50505); SEM = standard error of the mean.

Although specific embodiments have been illustrated and described herein, it will be appreciated by those of ordinary skill in the art that any arrangement that is calculated to achieve the same purpose may be substituted for the specific embodiments shown. This application is intended to cover any adaptations or variations that operate according to the principles of the invention as described. Therefore, it is intended that this invention be limited only by the claims and the equivalents thereof. The disclosures of patents, references and publications cited in the application are incorporated by reference in their entirety herein.

### BIBLIOGRAPHY

Bennett, J. W. and M. Klich. 2003. Mycotoxins. Clin. Microbiol. Rev. 16(3):497-516.
Bondy, G. S. and J. J. Pestka. 2000. Immunomodulation by fungal toxins. J. Toxicol. Environ. Health B Crit. Rev. 3(2):109-43.
Bouhet, S. and I. P. Oswald. 2005. The effects of mycotoxins, fungal food contaminants, on the intestinal epithelial cell-derived innate immune response. Vet. Immunol. Immunopathol. 108:199-209.
He, P., L. G. Young, and C. Forsberg. 1993. Microbially detoxified vomitoxin-contaminated corn for young pigs. J. Anim. Sci. 71:963-967.
Osweiler, G. D. 2006. Occurrence of mycotoxins in grains and feeds. In Diseases of Swine, 9^{th} ed., ed. B. E. Straw, J. J. Zimmerman, S. D'Allaire, and D. J. Taylor, 915-929. Ames, Iowa: Blackwell Publishing.
Pestka, J. J. 2003. Deoxynivalenol-induced IgA production and IgA nephropathy-aberrant mucosal immune response with systemic repercussions. Toxicol. Lett. (140-141):287-295.
Spurlock, M. E. 1997. Regulation of metabolism and growth during immune challenge: an overview of cytokine function. J. Anim. Sci. 75:1773-1783

What is claimed is:

1. An isolated Bacillus strain wherein the Bacillus strain is selected from the group consisting of B. licheniformis 3-12a (NRRL B-50504), B. subtilis 4-7d (NRRL B-50505), B. licheniformis 4-2a (NRRL B-50506), and B. subtilis 3-5h (NRRL B-50507), strains having all the characteristics thereof, any derivative or variant thereof, and mixtures thereof.

2. The isolated Bacillus strain of claim 1, wherein the strain is capable of detoxifying one or more mycotoxin(s) by a least one of the following: (a) at least about 60% when the one or more mycotoxin(s) is exposed to the strain in a viable state or (b) at least about 30% when the one or more mycotoxin(s) is exposed to supernatant of the strain.

3. The isolated Bacillus strain of claim 2, wherein the mycotoxin(s) is(are) selected from the group consisting of trichothecene(s), aflatoxin(s), citrinin(s), ochratoxin(s), and zearalenone(s), and combinations thereof.

4. The isolated Bacillus strain of claim 2, wherein the mycotoxin(s) is(are) selected from the group consisting of T-2 toxin, diacetoxyscirpenol (DAS), fumonisin(s) and deoxynivalenol (DON), and combinations thereof.

5. The isolated Bacillus strain of claim 2, wherein the mycotoxin(s) is deoxynivalenol (DON).

6. The isolated Bacillus strain of claim 2, wherein the detoxification is through biotransformation of the mycotoxin(s) to a compound with reduced toxicity.

7. The isolated Bacillus strain of claim 1, wherein the Bacillus strain is B. licheniformis 3-12a (NRRL B-50504).

8. The isolated Bacillus strain of claim 1, wherein the Bacillus strain is B. subtilis 4-7d (NRRL B-50505).

9. The isolated Bacillus strain of claim 1, wherein the Bacillus strain is B. licheniformis 4-2a (NRRL B-50506).

10. The isolated Bacillus strain of claim 1, wherein the Bacillus strain is B. subtilis 3-5h (NRRL B-50507).

11. A composition comprising supernatant from one or more culture(s) of one or more strain(s) from claim 1.

12. An isolated strain capable of alleviating a gastrointestinal inflammatory response when the strain is administered to an animal, wherein the strain is a lactic acid bacteria strain or a Bacillus strain.

13. The strain of claim 12, wherein the strain reduces the gastrointestinal inflammatory response by at least an about 2-fold reduction in gene expression of inflammatory cytokines TNF-a and MIP-2 in intestinal cells or tissue of an animal receiving the strain when the strain is administered to an animal.

14. The strain of claim 12, wherein the lactic acid bacteria strain is a Lactobacillus strain.

15. The strain of claim 12, wherein the lactic acid bacteria strain is L. johnsonii PLC B6 (NRRL B-50518), Enterococcus faecium 2-1d (NRRL B-50519), strains having all the characteristics thereof, any derivative or variant thereof, and mixtures thereof.

16. The strain of claim 12, wherein the Bacillus strain is of B. licheniformis 3-12a (NRRL B-50504), B. subtilis 3-5h (NRRL B-50507), strains having all the characteristics thereof, any derivative or variant thereof, and mixtures thereof.

17. A composition comprising supernatant from one or more culture(s) of one or more strain(s) capable of alleviating a gastrointestinal inflammatory response when the strain is administered to an animal, wherein the strain is a lactic acid bacteria strain or a Bacillus strain.

18. The composition of claim 17, wherein the lactic acid bacteria strain is a Lactobacillus strain.

19. The composition of claim 17, wherein the lactic acid bacteria strain is L. johnsonii PLC B6 (NRRL B-50518), Enterococcus faecium 2-1d (NRRL B-50519), strains having all the characteristics thereof, any derivative or variant thereof, and mixtures thereof.

20. The composition of claim 17, wherein the Bacillus strain is of B. licheniformis 3-12a (NRRL B-50504), B. subtilis 3-5h (NRRL B-50507), strains having all the characteristics thereof, any derivative or variant thereof, and mixtures thereof.

21. A composition comprising Lactobacillus brevis 1E-1 (ATCC PTA-6509) and one or more strain(s) selected from the group consisting of B. licheniformis 3-12a (NRRL B-50504), B. subtilis 4-7d (NRRL B-50505), B. licheniformis 4-2a (NRRL B-50506), and B. subtilis 3-5h (NRRL B-50507), L. johnsonii PLC B6 (NRRL B-50518), Enterococcus faecium 2-1d (NRRL B-50519), strains having all the characteristics thereof, any derivative or variant thereof, and mixtures thereof.
